(19) United States
(12) Patent Application Publication
(10) Pub. No.: US 2010/0191475 A1
(43) Pub. Date: Jul. 29, 2010

(54) METHOD FOR IDENTIFICATION AND FUNCTIONAL CHARACTERIZATION OF AGENTS WHICH MODULATE ION CHANNEL ACTIVITY

(76) Inventors: Scott Perschke, Glen Rock, PA (US); Ming Liu, Rockville, MD (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/717,503

(22) Filed: Mar. 4, 2010

Related U.S. Application Data
(63) Continuation of application No. 11/303,603, filed on Dec. 16, 2005, now abandoned.
(60) Provisional application No. 60/636,494, filed on Dec. 16, 2004.

(51) Int. Cl. G06F 19/00; C07D 451/12; C07D 223/28; C07D 307/81; C07D 453/04; C07C 311/08; C07D 405/14; C07D 401/12; C07D 211/14; C07C 217/48; C07D 211/22; C07H 17/08; C07D 403/04; C07D 211/52; C07D 487/04; C07D 211/60; C07C 255/43; C07D 211/58; C07C 217/64; C07C 211/63

(52) U.S. Cl. 702/19; 546/130; 540/592; 549/467; 546/134; 564/81; 544/370; 546/199; 546/203; 564/317; 546/241; 536/7.2; 540/557; 546/217; 544/282; 546/201; 546/228; 558/426; 546/221; 564/352; 564/289

(57) ABSTRACT

Materials, methods and a computer system are provided which facilitate the identification and characterization of modulators of potassium ion channels, particularly the HERG channel.

This application claims priority to U.S. provisional Application, 60/636,494 filed Dec. 16, 2004, the entire contents of which are incorporated by reference herein.

### FIELD OF THE INVENTION

The present invention relates to the fields of pharmacology and rational drug design. More specifically, the invention provides methods for identifying agents which modulate ion channel activity, a database of agents so characterized and computer software programs for further assessing potential therapeutic compounds which contain common structural and/or biophysical characteristics. In one aspect, such compounds are assessed for deleterious effects against specific ion channels, particularly the HERG potassium channel.

### BACKGROUND OF THE INVENTION

Several publications and patent documents are cited throughout the specification in order to describe the state of the art to which this invention pertains. Each of these citations is incorporated by reference herein as though set forth in full.

The HERG (human ether-a-go-go-related) gene encodes a membrane protein that functions as a K⁺-channel. This channel participates in the repolarization of cardiac tissue. A delay in repolarization is related to cardiac arrhythmias and heart attack. Inhibition of potassium flux through the HERG channel is associated with prolongation of the QT interval (Long QT; part of an EKG trace), i.e. delayed repolarization. These delays are associated with both bradycardia and arrhythmia. Therapeutic agents having diverse chemical structures have been associated with LQT and/or are suspected of causing adverse interactions with HERG protein. Examples of these different classes of drugs include the following: non-sedating antihistamines (astemizole, terfenadine), macrolide antibiotics (erythromycin) quinolone antibiotics (sparfloxacin), antipsychotics (haloperidol, clozapine, pimozide), prokinetics (cisapride), antiarrhythmics (dofetilide), non-potassium cationic channel blockers (verapamil, quinidine), beta-adrenergic blockers (sotalol), anti-fungals (ketoconazole), antimalarials (mefloquine, halofantrine), and biogenic amine transport inhibitors (imipramine, cocaine). Natural peptide toxins (ergtoxin, Bekm-1) from scorpions (both old and new-world) have recently been identified as potent and specific inhibitors of HERG. There are also reports that cAMP alters HERG activity by interaction at a cyclic nucleotide-binding domain (63). Exemplary pharmaceutical agents having demonstrable adverse HERG effects include for example, dofetilide (Tikosyn®), cisapride (Propulsid®), terfenadine (Seldane®), and astemizole (Hismanal®). These agents have been removed from the marketplace due to adverse side effects associated with HERG interactions. Cisapride alone is reported to be responsible for some 80 heart attacks and >300 hospitalizations (www.propulsid-eresource.com/what.cfm). Such removal of previously approved drugs from the market or drug candidates in developmental pipelines is costing the industry billions in revenues and hundreds of millions in research, development and legal costs.

It is clear from the foregoing that agents which adversely interact with HERG have the potential to cause serious damage or death. Accordingly, the FDA is expected to release guidelines in the near future requiring some measure of HERG data with Investigational New Drug submissions. In order to avoid such deleterious effects and eliminate safety concerns, drug manufacturers' require robust and readily available testing methods to assess such candidates and eliminate them from the development pipeline.

### SUMMARY OF THE INVENTION

In accordance with the present invention, in silico screening methods for identifying test compounds which modulate potassium channel activity are provided. An exemplary method entails assembling a dataset of agents known to modulate potassium channel activity, the dataset containing biophysical and structural features of such agents which include observed biological effects of such agents on potassium channel activity; providing a series of algorithms which describe the interaction of the structural features described above with the potassium channel; and assessing the test compound for the presence or absence of these structural features using algorithms described herein, thereby identifying test compounds sharing structural features with said agents which also modulate potassium channel activity. Also encompassed by the invention are test compounds identified by the foregoing method. In a particularly preferred embodiment, the potassium channel is the HERG protein channel and the method is performed to identify test compounds which may exhibit deleterious interactions with the HERG protein.

Another aspect of the method of the invention, entails contacting HERG expressing cells with any test compound identified in the initial in silico screening method and determining the effects of the test compound on HERG channel function as compared to i) cells which do not express HERG; ii) HERG expressing cells which had not been exposed to said test compound; and iii) HERG expressing cells exposed to an agent known to modulate HERG. The method may further include detectably labeling any test compounds identified in the initial in silico screen and conducting in vitro binding assays to determine the binding affinity and the binding site of the compound for the HERG protein. Once functionally characterized, any data obtained using the foregoing methods can be included in the dataset of agents known to interact with potassium channels, (e.g., the HERG channel) for use in the in silico screening method described above.

In yet another aspect of the invention, a computer system for performing the method described above is provided. The computer system includes a first dataset of the biophysical and structural features of known agents which interact with potassium channels, including but not limited to the potassium channels listed in Table 4. In a preferred embodiment, agents which interact with the HERG channel will be identified. The computer system can further comprise a second data base which includes at least one database selected from the group consisting of a three-dimensional structure database, a sequence mutation database, a failed drug database, a natural product database, and a chemical registry database. Also included in the computer system of the invention is a program containing at least one algorithm for performing the in silico screening method described.

Finally, a new binding site on the HERG protein has been identified and is referred to herein as the E-4031 site. Thus, another aspect of the invention includes a functional cell based assay for identifying test compounds suspected of modulating HERG protein activity via interaction at the E4031 site. One such method comprises contacting HERG expressing cells with the test compound and determining the effects of the test compound on HERG channel function as compared to i) cells which do not express HERG; ii) HERG expressing cells which had not been exposed to said test compound; and iii) cells exposed to E4031. An in vitro assay for determining a test compound's binding affinity for the E-4031 site on HERG protein or a fragment thereof is also provided.

In a further aspect of the invention, kits for performing the screening methods at the E4031 site are disclosed. An exemplary kit includes HERG expressing cells, non-HERG expressing cells; reagents suitable for performing functional assays in whole cells; and optionally, reagents suitable for performing in vitro binding assays.

### BRIEF DESCRIPTION OF THE DRAWINGS

FIG. 1. a) HERG-transfected cells demonstrate dose dependent specific binding of [³H]-astemizole. B)Boiling of the HERG-CHO membranes denatures the protein, thereby reducing specific binding.

FIG. 2. Association over time of [³H]-astemizole with the HERG protein, as expressed in CHO membranes. Ymax=maximum DPM bound. K=association constant; HalfLife is time (in minutes) to achieve ½ of total equilibrium binding.

FIG. 3. Inhibition of [³H]-astemizole binding to HERG-CHO membranes by various compounds.

FIG. 4. Saturation of [³H]-astemizole binding to HERG-CHO membranes. Nonspecific binding was defined as that remaining in the presence of 10 μM terfenadine.

FIG. 5. An astemizole dose dependent block of the HERG K+ channel. Using this technique, one can follow the efflux of Rb+ into the supernatant. Rubidium is used because it flows through the HERG K+ channel, yet is not present in measurable quantities in regular media/water.

FIG. 6. Time course of Rb+ efflux from HERG-transfected CHO cells, using atomic absorption to detect channel function. Sensitivity to astemizole is also demonstrated.

FIG. 7. Dose responses of select compounds from the training library tested in the atomic adsorption (AA) functional assay. Full, partial and inactive inhibitors are included.

FIG. 8. Results of screening 26 compounds in the [³H]-astemizole binding assay, and the membrane potential dye and AA functional assays. Compounds were tested in duplicate at 10 μM, except for BeKm-1 and Ergtoxin, (0.1 μM), and astemizole (1 μM). Most of these compounds have been reported to inhibit the HERG potassium channel in patch clamp assays, and represent diverse therapeutic and chemical classes. Some compounds (E-4031 (800%), terfenadine (200%), and pimozide, sertindole, clofilium (1000%) showed apparent inhibition much greater than controls in the fluorescent dye assay.

FIG. 9. Comparison within each assay of predicted vs. experimental inhibition, by compound (10 μM). The accuracy of the binding assay is apparent in this presentation.

FIG. 10. Regression plots of experimental vs. predicted inhibition (10 μM) in each of the three assays.

FIG. 11. This figure compares the results of predictive in silico screening with the actual in vitro screening. Using an array of QSAR models, 18 compounds (from a set of 2,000 compounds) were predicted to be active against HERG K+ channel and 29 were predicted to be inactive. All 47 compounds were tested for HERG activity using [3H]-astemizole binding assay. 14 (of 18) were confirmed to be active; whereas 28 (of 29) were confirmed to be inactive. HERG_INH_EXP is a plot of the experimentally derived inhibition. QSAR_PREDIC is the inhibition predicted from the QSAR model. Each compound is color-coded. A horizontal line indicates perfect agreement between actual and predicted.

FIG. 12. This is a representation of “nodes or leaves” indicating the separation of compounds according to descriptors and activity association

FIG. 13. a) Plots of 406 compounds selected from in silico models for inhibition of binding to D1 (X-axis) vs. inhibition at other similar GPCRs. “g” is D1 vs. D1. B) Nine compounds identified from the 406 that have nearly complete selectivity for D1 over other similar receptors.

FIG. 14. Overlays of five HERG inhibitors (GBR 12909 marked in green; GBR12935 in white; terfenadine in red; pimozide in grey, and clofilium in blue) showing proximity of certain structural elements.

FIG. 15. Overlay of E-4031 (white), sotalol (blue) and MK-499 (grey), showing structural elements that differ from the compounds in FIG. 14.

FIG. 16. Example of genetic algorithm software in operation with QSARIS.

FIG. 17. This figure illustrates the method (combination of algorithms) used for the prediction of potential binding inhibition at the astemizole site on the HERG K+-channel. Each circle “indicates” an algorithm based on a set of chemical descriptors and their ability to forecast chemical affinities for the binding site. When all of the algorithms are combined, a consensus allows a more accurate prediction of potential positive candidates.

FIG. 18. Molecular characteristics of the 7030 compounds in a diversity library.

FIG. 19. FIGS. 19a) to 19e) show the medichem-rule and filters used to select the compounds of FIG. 18.

### DETAILED DESCRIPTION OF THE INVENTION

The present invention provides a computer system and in silico screening method for the rational design of agents or therapeutic compounds which modulate potassium ion channel activity. The HERG potassium channel is exemplified herein. We focused our efforts on the HERG protein because of previous reports indicating that adverse drug reactions with the HERG channel are associated with serious health consequences, including heart attack and death. Drugs that appeared to be otherwise effective and safe have been withdrawn from the market due to deaths associated with HERG channel blockage. Propulsid (cisapride) was withdrawn from the market in

July 2000 due to 80 deaths and 340 reports of heartbeat irregularities. Two newer and more popular antihistamines Hismanal® and Seldane® (astemizole, and terfenadine, respectively) were also pulled off the market due to dangerous interactions with HERG. Understandably, there is an increasing demand for methodologies that will allow prediction and identification of compounds with the potential to adversely impact HERG channel activity early in the drug discovery process. Such methods and assessment systems are provided herein.

Initially, we designed an array of in vitro assays which are more accessible and amenable to high throughput than those currently in use (e.g., patch-clamp). We then used these assays to generate a high quality dataset to facilitate the ability to forecast potential HERG interactions. The divergent structures of the chemicals that have been shown to interact with HERG suggests that inhibition of HERG-mediated potassium flux is mediated by interactions which occur at divergent sites on the protein. Published evidence exists on a small number of these drugs showing that they likely bind to an intracellular site of the HERG channel (10, 64). Literature on the peptide toxins indicates that they bind to the extracellular vestibule of the channel (3-5), while other drugs are reported to recognize sites inside the channel pore (57, 65). Clearly, analysis methods which include assessment of binding on multiple sites on the protein are highly desirable.

The presence of multiple small molecule binding sites on a single ion channel is common. L-type calcium channels bind benzothiazepines, dihydropyridines and phenylalkylamines at different sites (6-11, 50-51). Drugs that influence the GABA-A receptor /chloride channel complex interact at multiple sites (67, 68). There are as many as 6 sites that modulate sodium channels (66). The HERG channel apparently shares this multiple-site regulation feature. Using parallel cell functional assays and radiolabeled ligands, we identified and further characterized these different small molecule binding sites.

Measurements obtained from radioligand binding assays directly correlate the small molecular and physical chemical characteristics of the compound being assessed (charge distribution, shape and size, solubility, etc.) with its specific interacting environment within a specific site of a binding site, i.e. a biological target. The advantage and ability to assess specific bi-molecular interactions at a defined site and “environment” enables the development of a highly congruent dataset with which one may derive robust structure-activity relationships. The data provided by binding assays provides the basis for a highly reliable and robust QSAR that mathematically correlates chemical descriptors (“features” of a small organic molecule) with the observed biological activity. Cell based functional assays provide “global” assessment of chemical interference, providing further “in vivo” information to augment that obtained from in vitro binding experiments. An observed functional response confirms whether a “specific binding event” indeed delivers a cellular consequence and also is reflective of chemical interactions at all possible sites. Therefore, cell based functional assay have also been employed the confirm results obtained in the binding assays which in turn facilitate further characterization of the different small molecular binding sites present on the HERG channel. Binding studies coupled with cell based functional assays performed in parallel, should reveal all of these possible binding sites.

### Definitions:

The phrase “potassium ion channel” as used herein refers the most common type of ion channel. They form potassium-selective pores that span cell membranes. Potassium channels are found in most cells, and control the electrical excitability of the cell membrane. In neurons, they shape action potentials and set the resting membrane potential. They regulate cellular processes such as the secretion of hormones, so their malfunction can lead to diseases. Certain potassium channels are voltage-gated ion channels that open or close in response to changes in the transmembrane voltage. They can also open in response to the presence of calcium ions or other signalling molecules. Others are constitutively open or possess high basal activation, such as the resting potassium channels that set the negative membrane potential of neurons. When open, they allow potassium ions to cross the membrane at a rate which is nearly as fast as their diffusion through bulk water. There are over 80 mammalian genes that encode potassium channel subunits. The pore-forming subunits of potassium channels have a homo- or heterotetrameric arrangement. Four subunits are arranged around a central pore. All potassium channel subunits have a distinctive pore-loop structure that lines the top of the pore and is responsible for potassium selectivity. A list of exemplary potassium channels, including the HERG channel, is provided in the Table 4.

The phrase “in silico screening method” refers to a computer-based analysis method for screening and identifying agents which specifically interact with particular sites on a potassium ion channel, the HERG channel being exemplified herein.

The phrase “biophysical and structural features” includes those chemical and physical features attributable to the test compound being analyzed. These include, without limitation, molecular weight, solubility, hydrophobicity, hydrophilicity, atom type, 3D molecular moment, primary structure, secondary structure, tertiary structure and chemical functionalities etc. “Biological effects” as used herein includes, for example, modulation in potassium flux, agonist activity, antagonist activity, alterations in membrane potential, membrane depolarization, absence of interaction with the potassium channel under investigation, and channel blockage.

The phrase “adverse biological effects” as used herein refers to those effects associated with dysfunctional potassium flux. These include, without limitation, cardiac arrhythmia, bradycardia, heart attack, dementia and death.

As set forth in Example I, we have (1) developed an array of readily accessible in vitro assays; (2) identified multiple possible small molecular binding sites on the HERG protein; (3) generated a reliable dataset and (4) tested the feasibility of in silico forecasting of compounds suspected of adversely interacting with HERG. These results are disclosed herein below.

The following examples are provided to illustrate certain embodiments of the invention. They are not intended to limit the invention in any way.

The materials and methods set forth below are provided to facilitate the practice of Examples I and II.

### Example 1

Recombinant cell-line and cell culture for membrane preparations—We purchased a recombinant CHO cell line expressing the HERG protein from Albert Einstein Medical College (Dr. Thomas MacDonald). The HERG-CHO cells were grown under standard culture conditions in media containing Ham's F-12, 10% FBS, 1 mg/ml G418 and 2 mM L-glutamine. The cells were split 3 times a week at a ratio of 1:30. Cells were harvested using a freeze-thaw (−20° C. to 37° C.) cycle to release them from the surface to which they adhere, then centrifuged (2000G, 10 min. 4° C.) to afford the biomass pellet. The cells were then stored in −80° C. until use.

Membrane preparations and ligand binding assays—Frozen cell pellets were first thawed and homogenized in 10 to 20 ml of assay buffer. An aliquot was taken for protein determination and the remaining homogenate was centrifuged (48,000×g, 10 min., 4° C.). According to the determined protein concentration, the resultant pellet was suspended in Heylen's buffer and added to radioligand and test compound. The composition of Heylen's buffer is 20 mM HEPES, 118 mM NaCl, 50 mM L-glutamate, 20 mM L-aspartate, 11 mM glucose, 4 mM KCl, 1.2 mM MgCl₂, 1.2 mM NaH₂PO₄, 14 mM heptanoic acid, and 0.1% BSA, pH 7.4. After 30-45 minutes of incubation at ambient temperature, the assay suspensions were filtered over 0.1% PEI-treated GF/C filters and rinsed with 5 mis of cold 50 mM NaCl. Bound radioactivity was determined by liquid scintillation spectroscopy.

Sources of radioligand—Various different radioligands were used in order to identify candidates for a given binding site. A list of radiolabeled ligands utilized in Example 1, their commercial suppliers, type of radiolabels and corresponding catalogues numbers are given in Table 1.

TABLE 1

| ISOTOPE | LIGAND | CATALOG NO. | SOURCE |
|---|---|---|---|
| ³H | Astemizole | N/A Custom | Amersham |
| ³H | Haloperidol | NET-530 | PerkinElmer |
| ³H | Verapamil | NET-810 | PerkinElmer |
| ³H | D-888 | TRK-834 | Amersham |
| ³H | Quinidine | ART-542 | Amer. Radiochem. |
| ³H | WIN 35,428 | NET-1033 | PerkinElmer |
| ³H | Erythromycin | ARC-467 | Amer. Radiochem. |
| ¹⁴C | BeKm-1 LP | N/A custom | Amersham, LP method |
| ¹²⁵I | BeKm-1 BH | N/A custom | Amersham, BH method |
| ¹²⁵I | BeKm-1 | NEX-412 | PerkinElmer |

Cell functional assay using atomic absorption detection—Rubidium flux out of HERG-transfected CHO cells was characterized using a Shimadzu atomic absorption system. The amount of rubidium in the extracellular and intracellular compartments was determined after depolarization with 50 mM KCl, following a 3-minute incubation with test sample. The atomization buffer included 0.1% CsCl₂/1% HNO₃ to suppress ionization of rubidium.

Cell functional assay using membrane potential dye—A membrane potential dye-based functional assay based on the HERG-expressing CHO cells has been developed. This assay was performed on the same library in parallel with the radioligand and AA-based functional assay. HERG-expressing CHO cells were plated as for the AA assay, except they were loaded with 4 mM DiBAC₄ instead of RbCl. Test samples or controls were added inside a Molecular Devices FlexStation and readings were taken over a 25 minute time frame.

Membrane Potential Assay Procedure—100 uL of 250,000 cells/mL in media were added to a 96-well assay plate and cultured overnight. The cells were washed with Hanks/HEPES buffer with 2 g/L of glucose (loading buffer) and 100 uL of warmed loading buffer was added. 80 uL of the FLIPR Membrane Potential dye (Molecular Devices; dissolved in loading buffer) was then added and the samples incubated for at 45 min at 37° C. Drugs (10× final concentration) in loading buffer were run along with no-drug controls. Plates containing cells were placed into the fluorometer (warmed to 37° C.) and incubated for 2 minutes. 10× drug solution in 20 ul was added and fluorescence measured for 15 minutes to obtain maximum response. Maximum response plateau is expected at approximately 7 minutes. This value will be used for EC50 calculation. A FlexStation fluorometer with fluidics, kinetic capabilities, and excitation of 530 and emission of 565 nm is used, with a 550 nm emission cut-off. Typical HERG channel inhibitors such as cisapride (IC₅₀=45 nM) or dofetilide (IC₅₀=10 nM) will be used as controls (Tang et al., 2000). Test compounds within 3SD of the negative control will be considered inactive. For the other “actives”, IC₅₀ values will be determined in this assay and at 1 or 2 concentrations in the Rb⁺ flux assay.

Collection of test compound library and suppliers—In most cases, compounds that were chosen for the training library were selected based on reported interactions with HERG function and/or an association with LQT. Exceptions include GBR12909 and GBR12935, nicardipine, and propranolol, which have not been reported in literature as HERG active. See Table 2.

TABLE 2
Table 2 This list of 26 compounds was screened through all of the assays
described. All have been reported in literature to inhibit HERG function.
The cost for compounds 22 and 23 (BeKm-1 and Ergtoxin) prohibit testing
at 10 μM. However the reported Ki's for BeKm-1 and Ergtoxin inhibition
of HERG function are in the low nanomolar range. If they bind to the same
site as the radioligand, one would expect some inhibition at the tested
concentration of 100 nM. None was seen.

| | Drug | source | cat# | CAS# | MW | Test Conc., uM | References |
|---|---|---|---|---|---|---|---|
| 1 | Cocaine | SIGMA | C-5776 | 53-21-4 | 339.8 | 10 | 43-45 |
| 2 | GBR12909 | SIGMA | D-052 | 67469-78-7 | 523.5 | 10 | — |
| 3 | GBR12935 | SIGMA | G9659 | 67469-81-2 | 487.5 | 10 | — |
| 4 | Imipramine | RBI | I-111 | 113-52-0 | 316.9 | 10 | 47 |
| 5 | Amiodarone | RBI | A-119 | 1951-25-3 | 681.8 | 10 | 48-49 |
| 6 | E-4031 | Calbiochem | 324470 | 113558-89-7 | 510.5 | 10 | 53-54 |
| 7 | Quinidine | SIGMA | Q-0750 | 50-54-4 | 746.9 | 10 | 50-51 |
| 8 | (+/−)sotalol | SIGMA | S-0278 | 959-24-0 | 308.8 | 10 | 52 |
| 9 | ketoconazole | SIGMA | K1003 | 65277-42-1 | 531.4 | 10 | 46 |
| 10 | Astemizole | SIGMA | A6424 | 68844-77-9 | 458.6 | 10 | 56-59 |
| 11 | cyproheptadine | RBI | C-112 | 969-33-5 | 323.9 | 10 | 32-36 |
| 12 | diphenhydramine | SIGMA | D-3630 | 147-24-0 | 291.8 | 10 | 1-2 |
| 13 | Terfenadine | SIGMA | T-9652 | 50679-08-8 | 471.7 | 10 | 28-31 |
| 14 | erythromycin | SIGMA | E-6376 | 114-07-8 | 733.9 | 10 | 24-27 |
| 15 | Clozapine | SIGMA | C-6305 | 5786-21-0 | 326.8 | 10 | 23 |
| 16 | Haloperidol | SIGMA | H-1512 | 52-86-8 | 375.9 | 10 | 21-22 |
| 17 | Pimozide | RBI | P-100 | 2062-78-4 | 461.6 | 10 | 12, 16-20 |
| 18 | Risperidone | SIGMA | R-118 | 106266-06-2 | 410.5 | 10 | 12, 15 |
| 19 | Sertindole | Lundbeck | N/A | 106516-24-9 | 440.9 | 10 | 12-14 |
| 20 | Nicardipine | SIGMA | N-126 | 54527-84-3 | 516 | 10 | — |
| 21 | Verapamil | SIGMA | V-102 | 23313-68-0 | 491.1 | 10 | 6-11 |
| 22 | BeKm-1* | Alomone | RTB-470 | N/A | 4098 | 0.1 | 4-5 |
| 23 | Ergtoxin* | Alomone | RTE-450 | 8006-25-5 | 4738 | 0.1 | 3 |
| 24 | Cisapride | RDI | R-51619 | 81098-60-4 | 466 | 10 | 37-42 |
| 25 | Propranolol | SIGMA | P-128 | 3506-09-0 | 295.8 | 10 | 74-78 |
| 26 | Clofilium | SIGMA | C-128 | 92953-10-1 | 510.2 | 10 | 60 |

*Indicates natural peptide toxins.

QSAR modeling and software application—QSARIS v.1.2 (from SciVision-MDL) was the primary data interrogation tool. The training was conducted with the results from 23 compounds in [³]-astemizole radioligand binding assay (Table 2). The large protein toxins that were part of the initial library were not used in the training set, due to the disparity in size and structural components with the small molecule samples. The percent inhibition at 10⁻⁵M was used to define observed biological activity. Software provided more than 200 different chemical descriptors including atom type, 3D molecular moment, substructural and molecular properties. Different chemical descriptors were randomly combined and regression models were produced based on the statistical correlations between the combined descriptors and the observed activities. The models were then examined and validated based on (1) R²-coefficients, (2) cross-validation index and (3) P-test. Six models with R²≧0.9 also met the cross-validation (one randomly withheld) requirement. These six models were used in the in silico forecasting experiments.

### Result and Discussions:

Functional assays employing whole cells provide results which are more reflective of the “in vivo” condition than those obtained from in vitro binding assays. Functional assays provide information about the agonist and antagonist effects of interacting molecules on a receptor or an ion channel.

One whole cell based functional assay we employed was based on the voltage sensitive dye DiBac₄, using a detection method originally developed by Dr. Vince Groppi of Pharmacia-Upjohn FLIPR and FlexStation fluorescence detection systems. Cells expressing ion channels like HERG protein are hyperpolarized in the resting state. Inhibition of ion channel activities allows cells to return to normal potential. As the cell membrane becomes more positive, dye migrates into cell membrane, increasing the quantum efficiency of the dye and thus increasing fluorescence. For practical purpose, the fluorescent method is a “user-friendly assay” for its ease of operation, reproducibility and adaptability to high throughput formatting. Large number of compounds may be readily tested in either 96- or 384-well format. The mechanism of detection is based on the dye translocation in response to changes of the membrane environment. In certain circumstances, it may be desirable to perform confirmatory assays.

As an alternative and a parallel confirmative assay, the Rb-flux assay was employed using the methodology reported by Tang (Tang et al, 2001). Minor modification of the published protocol was necessary due to different expression levels of the HERG protein in recombinant cells. Astemizole, terfenadine, pimozide and haloperidol, which completely inhibited HERG channel activity, were used to validate this assay.

[³H]-astemizole was employed in our studies based on previous reports that this compound demonstrates high affinity (KD=3 nM) binding with HERG protein expressed on HEK-293 cells (Heylen 2002). This observed binding affinity is consistent with patch-clamp observations and in accordance with our internal observation from cell based functional assays using both membrane potential dye and Rb⁺ flux.

Two cell lines typically utilized to express HERG K+ channel are HEK293 and CHO. The use of CHO cells is exemplified herein. The CHO line is a relatively “clean” system (as opposed to the corresponding HEK cells). There is no endogenous ion “action” in the CHO cells that is similar to the ion flux that is controlled by the HERG protein. In the experimental system using HERG-CHO cells, the assessment of chemical interference or changes in K⁺ flux are the sole consequence of HERG protein activity. The HEK-293 line is more complicated. There is an Iₖᵣ-like ion flux in the native cells of HEK293. Reportedly, [³H ]-dofetilide, a drug known to be specifically reactive with HERG, also exhibits high affinity to a membrane component of the native cells of HEK-293 (Finlayson, 2001).

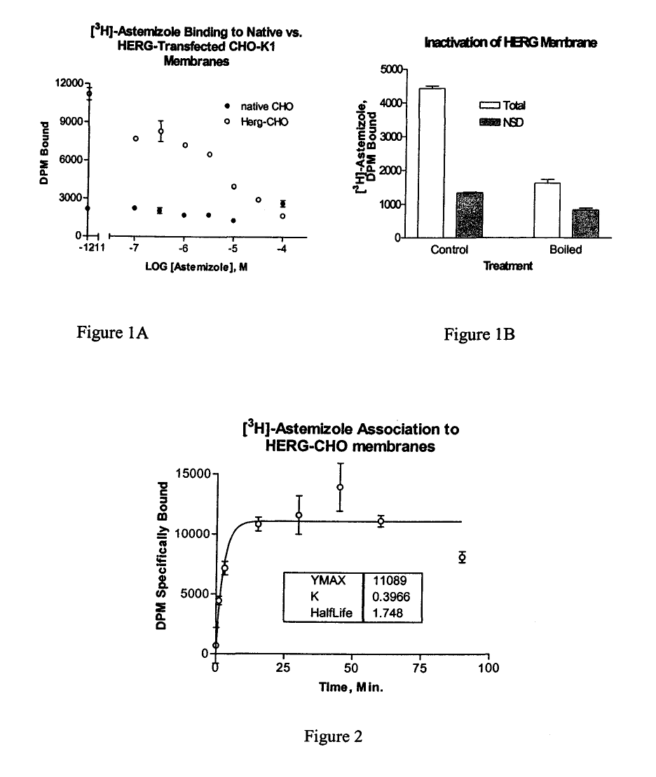

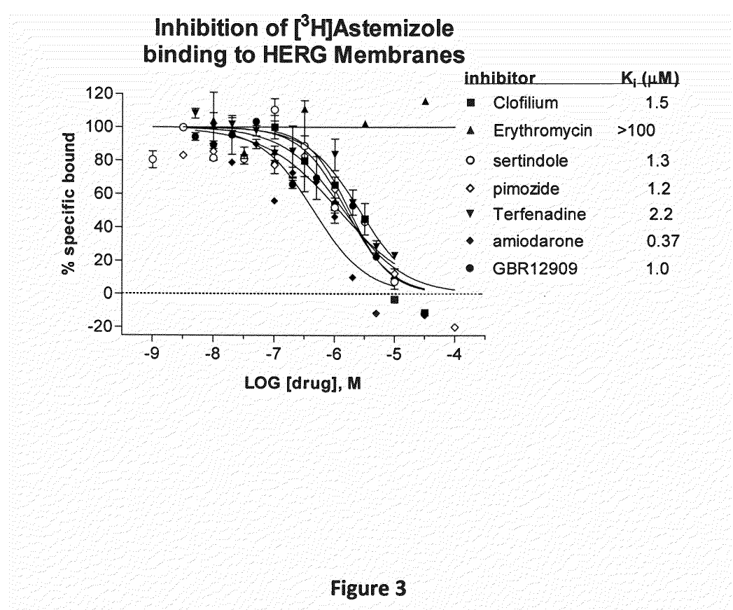

Wild-type and recombinant HERG-expressing CHO cells demonstrate a significant differential in [³H]-astemizole binding. As indicated in FIG. 1, the dose response curve confirmed the presence of binding specific to the HERG-transfected CHO cells. The control experiment demonstrated that denaturation of the target protein using heat (boiling), abolished the observed specific binding. Further experimental evidence, shown in FIG. 2, indicates that the interactions between [³H]-astemizole and the HERG protein occur at concentration and temperature dependent thermodynamic equilibrium. At the given protein concentration (25-50 μg/tube) and at ambient temperature, the time required for this interaction to reach the such an equilibrium is less than 12 minutes; hence incubation times of 30 to 60 minutes at ambient temperature were employed.

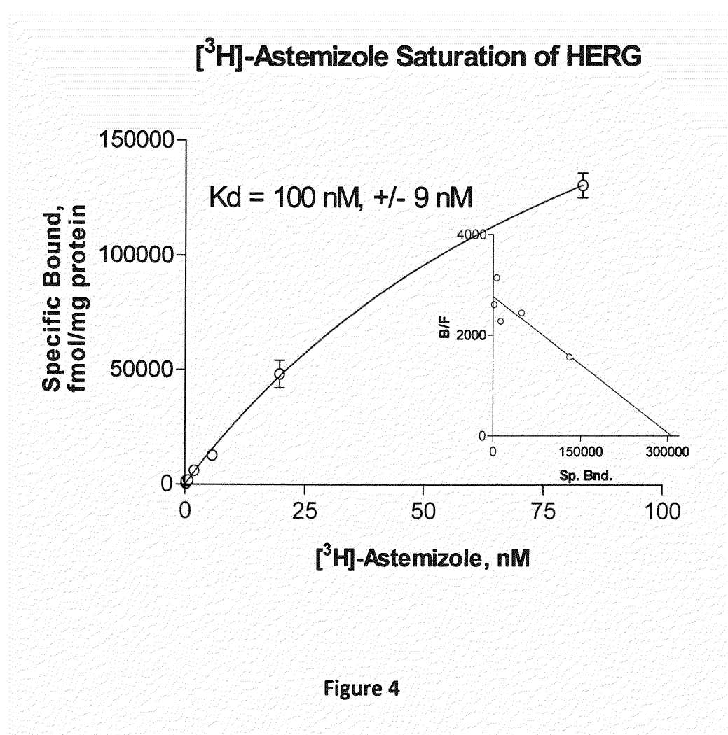

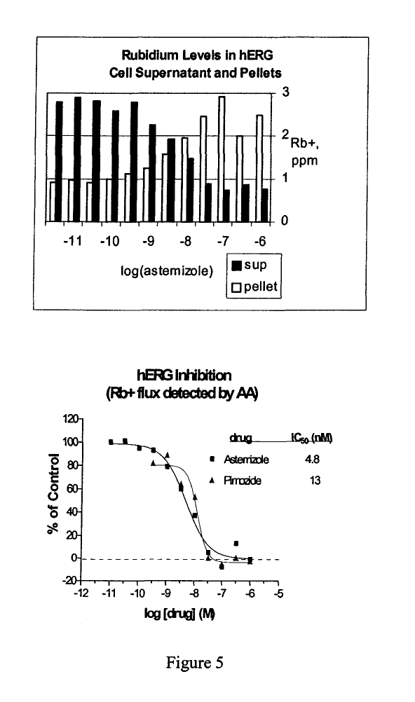

Pharmacological characterization of the [³H]-astemizole binding site was assessed using competitive binding experiments. Binding of [³H]-astemizole in the presences of 6 potential competitors, namely amiodarone, clofilium, erythromycin, pimozide, sertindole and terfenadine was determined. These assay results are shown in FIG. 3. We also performed experiments to determine the level at which binding of [³H]-astemizole became saturated. Twelve concentrations of [³H]-astemizole were used, ranging from 1 to 400 nM, under total and non-specific binding conditions. The results of the saturation studies are shown in FIG. 4.

In addition to [³H]-astemizole, we also tested the different radioligands listed in Table I. These compounds were chosen for their reported activity in causing LQT and for their availability in radiolabeled form. [³]-Haloperidol exhibits high binding levels with both the wild type and the recombinant CHO cells used for our assays. Blockers of haloperidol binding sites (spiperone to block dopaminergic, N-methylscopolamine to block muscarinic, prazosin and oxymetazoline to block a 1- and α2-adrenergic receptors, pentazocine to block sigma sites, and aconitine to block Na site 2 binding sites) failed to reveal a difference between native and transfected cells. This lack of a differential suggests that this particular radioligand is not ideal for assessing HERG interactions. Radiolabeled verapamil, D-888, quinidine, WIN-35428, and erythromycin were likewise tested. None of these compounds indicated sufficient specificity for the recombinant protein to qualify them as ligands in binding studies. We also did not observe sufficient binding with a custom preparation of the iodinated scorpion toxin, [¹²⁵]-BeKm-1. Although known to be HERG ion channel inhibitor, the iodination reaction used in this preparation of the toxin seems to have modified the amino acid residues that are required for binding. We have since obtained iodinated toxin from Perkin Elmer which worked well in our system. Recently obtained data revealed that terfenadine has moderate affinity for this site whereas cisapride has low affinity.

The Rb assay was developed using the methodology of Tang et al. A review of the literature indicated that astemizole is a high affinity, commonly used, commercially available ligand for HERG blockage. It also worked well in our HERG membrane potential dye assay. A typical report for astemizole IC50 is about 5 nM for patch clamping, 100 nM for membrane potential dye and 10 nM for atomic absorption.

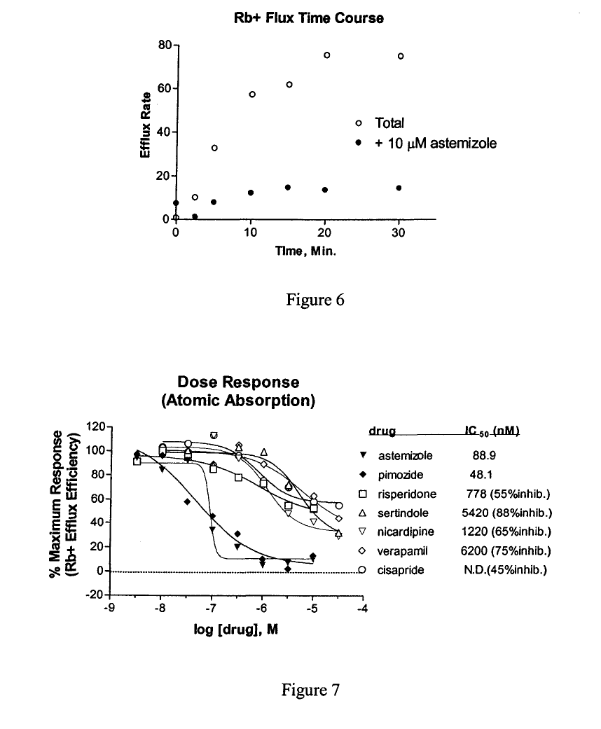

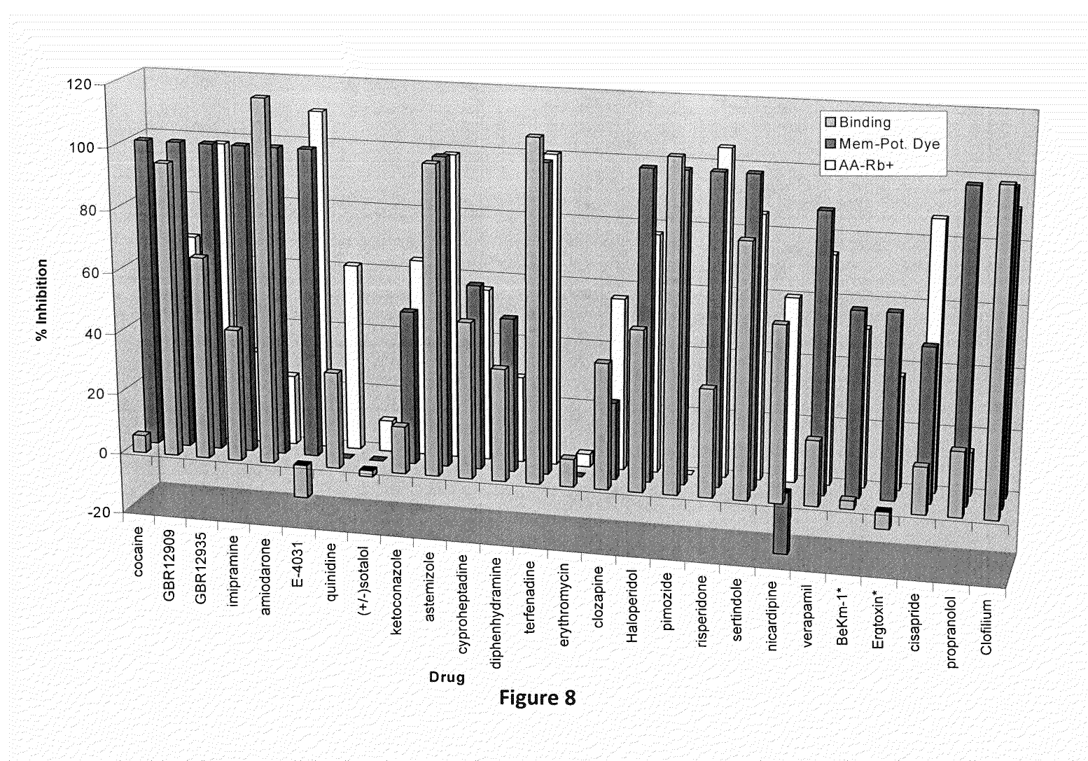

Initial experiments revealed that the multiple washings in the methods described by Tang caused cell loss and reduction of Rb inside the cell. We determined that one wash was sufficient and marginally better than no wash. To maintain sample sensitivity and to have enough sample to inject, a 1:1 dilution of sample with 0.1% CsCl/1% HNO₃ provides better sensitivity. A 1:2 dilution also works but at 1:3 our sensitivity became poor. Per the vendor's suggestion, we use 200 uL injections with appropriate wash steps, using detection of absorption peak. Two injections per sample are made into a Shimadzu AA and if the cv reaches 10%, a third injection is performed; the computer selects the two closer values. A cut off of 10% catches major errors and allows a reasonable analysis speed. A time course was performed, shown in FIG. 6. Rb+ efflux actively occurs from 0 to 30 minutes, thus 25 minutes was selected as an appropriate time point. An initial change due to astemizole addition was observed between 0 and 2.5 minutes. We therefore allow drugs to pre-incubate with the cells for 5 minutes. Adverse effects at 10 and 3% DMSO were noted, whereas 1% and less had no apparent effect. Therefore, DMSO is limited to <1%. See FIGS. 5 and 6.

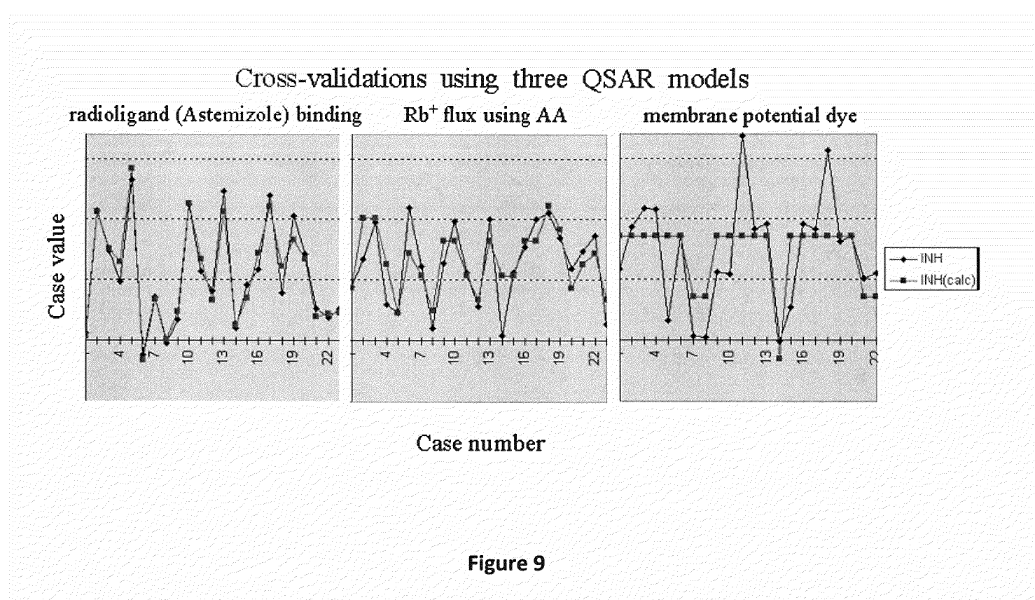

Dose response experiments were also performed (FIG. 7). Astemizole, terfenadine, pimozide and haloperidol completely inhibited the HERG channel. Other drugs such as cisapride provided partial block of the Rb+ efflux whereas some reported blockers such as propranolol, sotalol, imipramine, erythromycin and diphenhydramine showed no inhibition at up to 30 uM. Other compounds listed in Table 2 appear to be partial channel blockers.

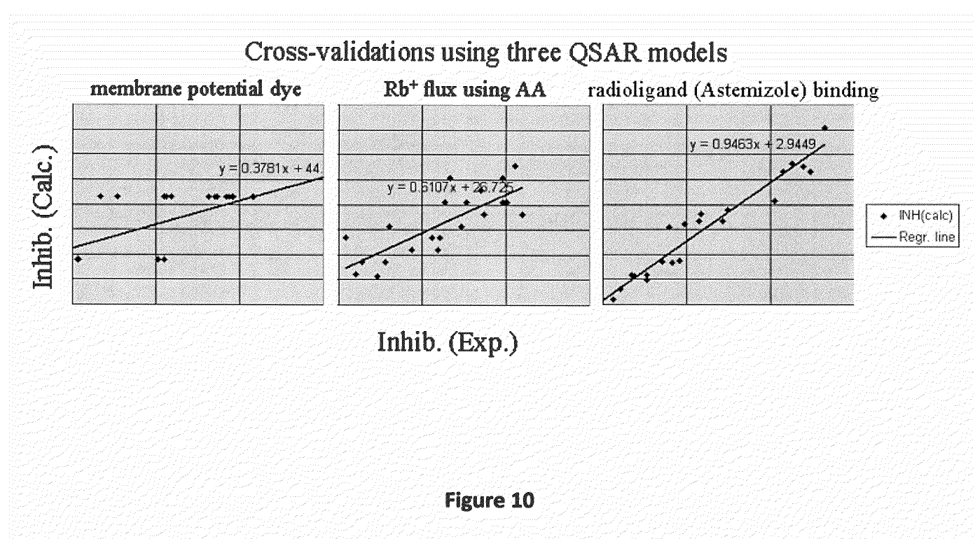

We tested this panel of compounds at 10⁻⁵M in these assays. The purpose of these experiments was to: (1) compare and cross-validate different assay formats; (2) use functional assays to provide additional indications of additional binding sites that are distinct from the [³H]-astemizole site; and (3) generate a small but congruent dataset, with which we can establish algorithms for forecasting potential activity (or more importantly the lack of activity). The compounds tested were selected according to their reported activities, either as class III antiarrhythmic medications (drugs that affect mainly K+movement, such as amiodarone, dofetilide, E-4031, sotalol etc), or for their reported clinically observed cardiac effect in QT-prolongation (such as terfenadine, cisapride, and astemizole, etc). The results obtained from testing this panel of compounds in three different assays using recombinant HERG-CHO cells are shown in FIG. 8.

For the most part, the results and observations from both cell based functional assays are consistent. There are four exceptions, namely quinidine (#7), (±)-sotalol (#8), erythromycin (#14) and nicardipine (#20). These four compounds initially did not exhibit any activity in the dye-based assay, and are only modestly active in the assay using atomic absorption. Each appears to be an exception from the norm.

A recent study indicated that the inhibitory actions of sotalol and erythromycin are markedly temperature dependent (Stanat, et al, 2003; Kirsch et al, 2004). Both dye- and atomic absorption based whole cell functional assays in our initial experiments were conducted at room temperature, a condition that is sub-optimum, which is the likely reason of the observed modest activity in atomic absorption assay and lack of activity observation in the dye-based assay.

Another recent report indicates that quinidine blockade of the ion channels is pH, voltage- and time-dependent. At positive membrane potentials, quinidine caused frequency-independent block mainly through this fast blocking kinetic (Tsujimae et al, 2004); moreover acidification weakens the inhibitory effects of quinidine on HERG channels (Dong et al 2004). The assay using the membrane potential dye as an indicator was conducted at a pH (˜7.2) which detected little measurable signal upon addition of quinidine, whereas under a similar condition but with a raised pH (˜7.6), a higher than 60% inhibitory activity was observed using atomic absorption detection. This change coincides with the published observations. Such pH dependency is also consistent with the SAR-QSAR observations. There is a propensity of forming intra-molecular hydrogen bond specifically which is negatively contributing to its affinity with the respected protein. Changes of pH may affect the H-bond formation, hence affecting the activity.

Nicardipine, a 1-4 dihydropyridine calcium antagonist and one of the first intravenous dihydropyridine calcium channel antagonist, at 30 mg/kg caused sustained hypotension and tachycardia in humans (Horii et al 2002) also lacked activity in the dye-based assay. However, there is yet not definitive data explaining the mechanism underlying HERG-nicardipine interaction. Yet, dose-dependently, it shortens QTrc and produced sinus arrest in both WT and TG mice (Lande et al, 2001). In another study, nicardipine (1 micro M) slightly, but significantly, shifted the voltage dependence of activation and steady-state inactivation to more negative potentials, and also slowed markedly the recovery from inactivation of Kv4.3L currents (Calmels, 2001; Hatano et al 2003); that is, the calcium channel inhibitor markedly affects hKv4.3 current, an effect which must be considered when evaluating transient outward potassium channel properties in native tissues. Thus, its cardiac effect appears to be due to a combination effect on the HERG and other K⁺-channel isoforms.

Certain incongruities between “binding” and “functional” measurements are not surprising. Binding of the radioligand to the target is a “local event”. A chemical interacting with the HERG protein at other than the [³H]-astemizole site may demonstrate weak of no observed affinity in a [³H]-astemizole binding assay. In contrast, functional assays do not have the same site restriction as do binding assays. Chemicals may react with the ion channel at any possible site thereby rendering a cellular response. In this dataset, both E-4031 and cisapride show limited effect in the binding assay (0˜15% inhibition), but strong functional responses (90˜100%). Thus, E-4031 and cisapride appear to represent ligands that are interacting with HERG protein at sites other than the astemizole binding site.

Amiodarone presents another idiosyncrasy. Amiodarone is known to be an efficacious proarrhythmic with minimal risk (as opposed to dofetilide and sotalol) of the class III anti-arrhythmics. It is also listed in other antiarrhythmic classifications (class I, Na⁺ channel; class II, β-blocker; class III, K⁺ channel; and class IV, Ca⁺⁺). Amiodarone is the only compound that exhibited significant binding affinity in the [³H]-astemizole/hERG assay that also lacked or had minimal activity in the functional assays. Such a discrepancy in experimental observations provides insight on the regulation of cardiac activities through multiple ion channels (Na⁺, K⁺ and Ca⁺⁺).

Using the chemical structures and the data obtained from these assays we established QSAR models. The purposes of this effort are two fold: (1) to determine whether the dataset generated by the assays is sufficiently “consistent and congruent” for QSAR development; and (2) whether these “relationships” are sufficiently useful in forecasting the potential presence and absence of hERG activity. Three models of activity were generated using the computational software QSARIS (a SciVison product). This computational program employs multiple regression analysis to link chemical descriptors with the observed biological activity. The versatility of this software program is that it provides a pre-set array of chemical descriptors ranging from sub-structural components to quantum mechanic parameters. These pre-set conditions make this program user-friendly. The disadvantage of the tool package is that it lacks the dynamic ability to handle diverse chemical sets and multiple (or heterogeneous) interactions (chemical interactions at different sites).

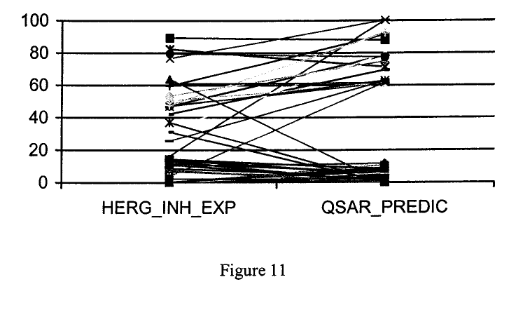

Table 3 tabulates QSAR models derived from the dataset for each of the three assays. All models are generated using a restricted set of chemical descriptors, e.g. sub-structural components. It is clearly shown that the radioligand binding assay generated the most congruent and internally consistent set of data. The regression models depict arrays of chemical descriptors prominently affecting activity at the HERG K+ channel. The binding assay model presented the highest regression quality, as reflected by the multiple R-squared and P values. The cross-validation (sequentially withholding one from the training set, and comparing the predicted values with the experimental values) experiments (results shown in FIG. 9) indicate that the constructed model could be used to predict potential interactions. Such a result is expected. A binding experiment is a direct measurement of bi-molecular interactions at a specific site, where the interacting descriptors (components of the micro- and macromolecules) are consistently reflected in the interacting affinities.

TABLE 3
Table 3 Statistical comparison of the preferred models for cross-validation of
hERG, based on training library data for each of the three assay methods.

| | | | Standard | | | | Cross |
|---|---|---|---|---|---|---|---|
| Data | Models (ordinary multiple regresion- | Multiple R- | error of | | | Multiple Q- | validation |
| Sources | descriptor_substructure) | Squared | estimation | F-statistic | P-value | Squared | RSS |
| Binding | INH = −11.26 * numHBa − 11.74 * SssO_acnt + | 0.9463 | 11.77 | 47.01 | 2.81E−09 | 0.8973 | 4243 |
| | 20.73 * SsF_acnt − 64.62 * SddssS_acnt + | | | | | | |
| | 12.26 * SHBint8_Acnt + 0.3362 * fw − 18.4559 | | | | | | |
| Dye | INH = −50.64 * SHBint3_Acnt + 86.0386 | 0.3781 | 36.63 | 12.16 | 2.32E−03 | 0.2737 | 3.14E+04 |
| Rb+ flux | INH = −29.6 * Ssl_acnt + 9.624 * SssCH2_acnt + 14.7308 | 0.6594 | 20.6 | 12.26 | 1.08E−04 | 0.2696 | 1.73E+04 |

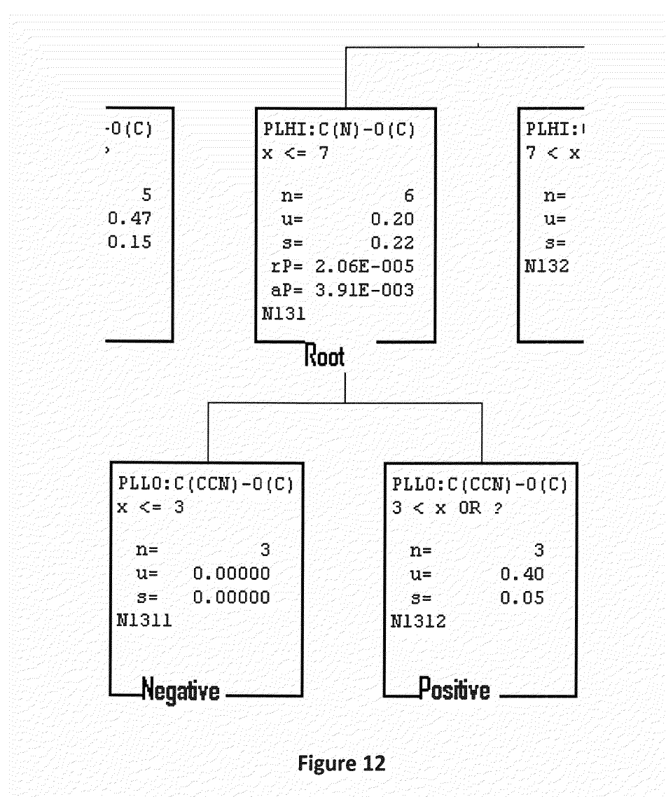

The data provided by the functional assays provided different results. With these data, the computation program could not depict a set of descriptors that are statistically and significantly linked to the observed biological activity. This result is also expected. QSAR modeling using regression models relies on specific molecular interactions, whereas the data provided by the functional assays likely reflects interactions at multiple sites. Notably, certain functional assays provide data of greater reliability than others. However, in the present study, the data obtained from in vitro binding assays generated the most congruent data set. The comparison of cross-validation using different models is shown in FIG. 10.

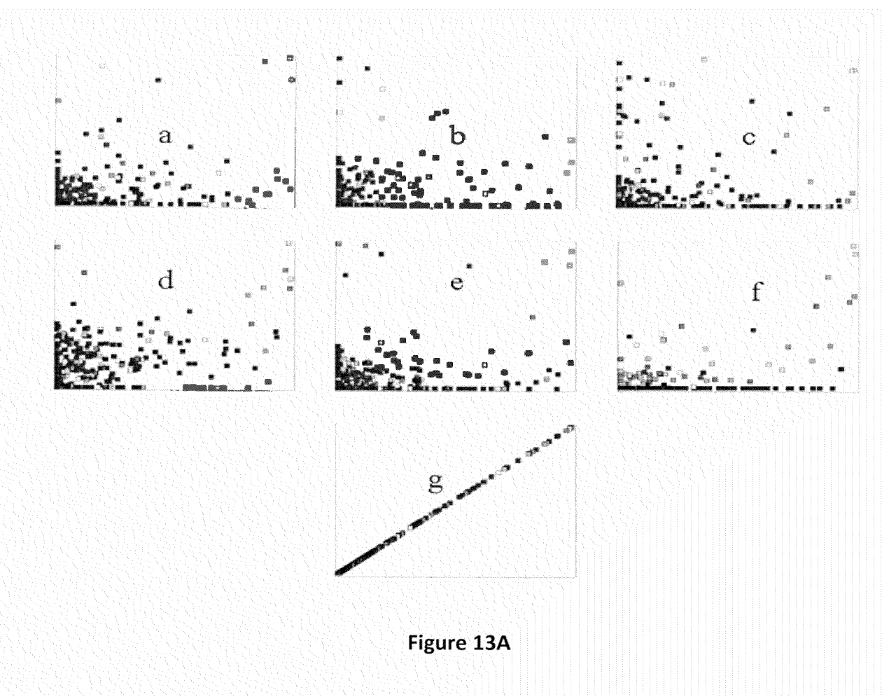

To test the validity (or the forecasting ability) of these QSAR model(s), we set up validation experiments. These experiments were designed to forecast or predict the activity of chemicals that are not in the training set, using the derived models, then testing the compounds (with predicted levels of activity) in the corresponding in vitro assay. The results of the validation experiment are given in FIG. 11. Using QSARIS, we generated multiple QSAR models based on the “binding dataset” and different sets of chemical descriptors. Various modules used substructural components, quantum mechanic parameters, chemical functionalities or through-bond distances. The structure-activity relationship is derived using multiple regressions between the observed binding activity and the set of chosen chemical descriptors. After some comparisons, it was determined that six models provided the best validation results.

These six models were used to scan a chemical library of 2000 compounds, mostly medications, assay reference agents, or other previously known bioactive compounds. Eighteen compounds indicated to be potentially reactive (predicted inhibition of ≧50%) with HERG protein using the six models. These compounds along with another 29 compounds (predicted to be inactive) were tested for activity in the [³H]-astemizole binding assay. Of the 18 compounds, 14 demonstrated greater that 50% inhibition, two were of modest activity and two were inactive. This result gives a 77.8 to 88.9% forecasting accuracy for compounds that are potentially active. Out of the 29 compounds predicted to be inactive, 1 demonstrated more than 50% activity and 2 demonstrated modest activity (20-40%). These limited results give a 90 to 96% forecasting accuracy for inactive compounds.

### Conclusion

We established multiple in vitro assays that can be used to readily assess changes in HERG K+channel activity as a consequence of chemical interactions with the protein. The pre-existing membrane potential dye and the novel radioligand binding assay are both amenable to high throughput screening, while the AA assay is highly consistent with patch clamp results. Using both functional and binding assays in parallel we have also gained further data indicating the presence of multiple binding sites on HERG.

We have also developed methods of forecasting potential interference of HERG K⁺ channel activity due to small molecule interactions. The results provided herein indicate that we can forecast potential activity related to the [³H-]astemizole and other binding sites.

### Example 2

Using the dataset obtained from the previous example, we found that the measurements obtained from a specific radioligand binding assay are largely but not completely compatible and similar to the measurements obtained from the Rb⁺flux assay. This observation is consistent with previously published experimental observations. We will employ multiple independent in vitro radioligand binding assays in combination with the high throughput membrane potential dye based and Rb⁺ flux characterization in order to reliably predict potential HERG liability, or the lack of it. After validation, these in vitro methods will provide readily available, easily accessible and inexpensive alternatives in vitro testing methods.

Using the dataset “discrepancies” between different assay results (binding vs. “functional”), we identified additional distinct small molecular binding site(s) on the HERG protein and ligand(s) that appear to be specific for these site(s).

We produced an array of robust mathematic algorithms capable of forecasting potential HERG K⁺ channel activities at the astemizole binding site. These algorithms, when used together, afford superior forecasting abilities that those previously published (Cavalli 2002, Ekins 2002). Our validation studies indicate that our forecasting ability to select compounds active at the astemizole binding site on the HERG K⁺ channel was about 90% and the ability to indicate that a compound is devoid of same approaches 100%. With an expanded dataset, we will generate a broader and more robust array of in silico prediction algorithms.

A large library of diverse chemical entities for HERG interaction using cell based functional assays will be screened. Firstly, the library comprising of a collection of more than 10,000 diverse chemicals representing 1.5 to 2 million chemical entities accessible commercially (and a collection of known ion channel ligands) will be screened for whole cell-based functional activity using high throughput methodology. Those possessing functional activity will be further tested for confirmation using additional and more stringent in vitro assays including atomic absorption, cell and tissue based patch-clamp methods. The results of this effort will be a large and highly (cross-) validated dataset comprising compounds which impact HERG K⁺-channel pharmacology. The library will then be expanded to include >150 (˜200) chemicals that were previously known to have ion channel activities (especially K⁺-channel), or chemicals that are structurally similar to those that are known active. By screening a large and diverse set of chemicals in multiple assays (functional/binding), we should identify all pharmacologically relevant small molecule binding sites on the HERG protein. Once the leads (screening hits) are found, the chemical library will then be further expanded to include those compounds that are structurally similar to the identified leads. These newly expanded and optimized library components will then be screened again in both functional and binding assays to detect potential activity.

As discussed in Example 1, there is strong evidence for multiple binding sites on HERG protein that are capable of modulating channel function. Ligands that recognize these sites (which are distinct from the astemizole binding site) will be custom radiolabeled and used to characterize these additional sites. We will initially focus on the E-4031 binding site and the peptide binding sites. However, all “hits” from Example 1 will be screened for activities in these assays. Idiosyncratic results, i.e., leads demonstrating “functional readings” but not “binding read-outs” in all of the three assays (astemizole, E-4031 and the peptide sites) will be labeled to explore new and additional binding domains thereby identifying as many as possible sites to which small molecules may bind to produce functional responses that are affecting K⁺-channel flux. These respective “sites” (marked by the respective labeled ligand) will be developed into individual binding assays.

Radioligand binding assays consist of 5 typical steps:

(1) Determination of appropriate concentration of protein to use in the assay. Ideally, one wants to assess binding in the linear range of protein concentration. Additionally it is desirable to minimize non-specific radioligand binding to the filters used in the assay. Seven different protein concentrations centered on 10 μg protein per tube (0.3 to 300 μg of total protein) are employed. To all tubes 10 nM of radiolabeled ligand is added. To the first 3 tubes of each set, vehicle is added to determine total binding. To the second 3 tubes of each set, 5 μM of the corresponding non-labeled (cold) ligand is added. The reaction is incubated for 2 hours, which should at least approach equilibrium. Counts from the tubes with non-labeled ligand define non-specific binding, hence the process (difference of first 3 tubes vs second 3) defines specific binding, and thus the ideal concentration of the protein used in the assay. This step will also be performed with native (non-transfected) CHO cells, to ensure that the native cells do not express detectable levels of the HERG channel.

(2) Equilibration Time—Time course experiments are conducted to determine the time to reach thermodynamic equilibrium (or steady state). Typically 0, 15, 30, 45, 60, 90, 120, and 150 minute time points are used. Normally the time course experiment is conducted at two temperature settings, ice (˜0° C.), ambient and/or 37° C. A dissociation assay will be performed on the second time course experiment to confirm reversibility of binding. Copious amounts (@1000-fold) of unlabelled ligand are added at various times (determined from the association experiment) to compete off the radiolabel from the binding site, after it has reached equilibrium.

(3) Saturation analysis—determines K_{D} and Bₘₐₓ. 12-16 different radioligand concentrations (the range for the proposed radioligands is 0.1 nM˜1,000 nM (approx. 3-4 conc/log unit) are used with a defined protein concentration, temperature and duration of incubation. Data from saturation experiments will be analyzed with a non-linear regression program (Graph-Pad Prizm, or similar) and plotted as a Saturation Isotherm with Scatchard graph inset. The second and third saturation experiments will be performed with the radioligand concentrations set to span 1 log unit higher and lower than the determined Kd value from the previous assay(s). Data will be analyzed and graphed using both non-linear and linear regressions. Non-linear regressions will be fitted to one and two site models to determine the better fit.

(4) Carrier effect—solvents used to solubilize samples (DMSO, ETOH) will be analyzed (in triplicate at final solvent concentrations of 0, 0.1, 0.4, 1, 4, and 10%) for effect on binding.

(5) Pharmacological characterization—As discussed previously at least 20 different compounds, shown in Table 2, are used to generate a matrixed (20×3) dataset. That is, the characterization will be accomplished by performing dose response analyses with 20 or more agents using 8 concentrations in triplicate covering a 4-log unit range. GraphPad's non-linear regression analysis will be used to determine IC₅₀ and Hill slope values from dose response experiments. Each curve will be fitted to 1 and 2-site models to determine the better fit. Inhibition constants (Ki) are derived from the IC₅₀ value via the Cheng-Prusoff equation (Cheng, Y. C. & Prusoff, W. H., 1973).

Potential effects from ions on binding will be tested by varying the concentrations of calcium, sodium and potassium in the assay buffer. Those concentrations that give the greatest level of specific binding will be used for screening assays.

The results obtained using the new binding assays and the expanded library collection of compounds will provide sufficient data density to derive robust modeling capability. This capability can be further expanded by screening compounds structurally clustered about those compounds that demonstrate potent activity. The result of this effort should provide a collection of chemicals balanced for their chemical diversity and convergence.

Based on the data obtained in the foregoing experiments, in silico screening algorithms have been developed to establish and validate a matrix of QSAR models. In silico screening software can also be developed to facilitate use of the algorithms provided herein. The matrix of the QSAR models is derived using the created database and is further based on the clusters of compounds demonstrating activities in the various binding assays.

Ion channels as important therapeutic targets for the treatment of a variety of disorders. The recent advances in our understanding of the human genome have revealed large numbers of K⁺-channel isoforms. In conjunction, advances in x-ray crystallography have also produced numbers of K⁺-channel models. The large numbers of K⁺-channels, their different tissue distributions, and biological/physiological functions provide new avenues for the development of pharmacologically important agents which modulate channel activity in a channel specific fasion.

Using our proprietary database, any chemical structure based data interrogation tools may be used for the SAR investigations. We frequently use recursive partitioning (RP; Chen et al, 1999; Rusinko, et al, 1999; 2002) and other computational software tools to interrogate the dataset and to derive structure activity relationships (and structure-inactivity-relationships). The advantage of RP is its ability to handle the co-existence of a multitude of structure-activity relationships (SARs), and the ability to sort and group these relationships accordingly. Moreover, this approach provides the ability to model and forecast nonlinear SARs, which are common phenomena when dealing with diverse chemical datasets and their respective interactions with macromolecules of multiple binding sites and orientation. One commercial software package useful for this type of analysis is ChemTree (GoldenHelix).

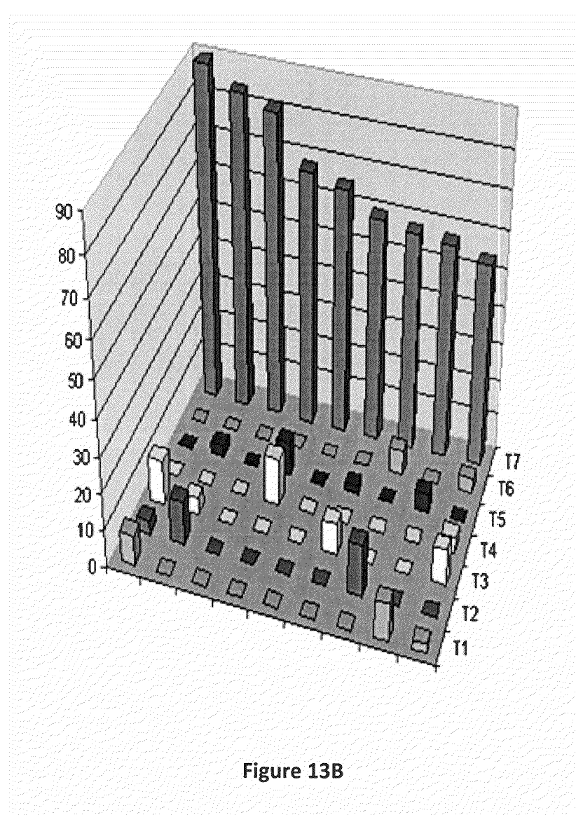

In general, statistical clustering is often superior and more versatile than other data handling algorithms. Such versatility is more pronounced when assessing “activity” data resulting from exposure to a diverse class of chemicals, multiple modes of activity (agonist, antagonist, partial agonist, inverse agonist etc), and different orientations of molecular interactions. The following discussion relates to data sets describing GPCR receptors. Chemical descriptors associated with a particular activity can be separated from those descriptors that are devoid the same activity. FIG. 12 represents a typical example of chemicals separated using recursive partitioning into containing descriptors associated (positive)/unassociated (negative) with a particular activity.

Using the descriptors associated with certain biological activities, increases the likelihood of finding active compounds with specified activities; whereas using descriptors devoid of such associations will likely lead to the identification of inactive compounds (against the target of interest). That is, one may use the positive descriptors to find compounds (from combinatorial library suppliers for instance) likely to interact with the specified target. The resultant list may then be sequentially “trimmed” with descriptors that are negative for statistical association with potential off target proteins or receptors. The subsequent and final list of compounds obtained from this analysis will be an enriched population of “activity biased” small molecules.

This “sequential in silico screening” approach will translate into a higher probability of finding compounds that are active against the receptor of interest and are inactive with non-target proteins Previously, we conducted a study to identify dopamine D₁ selective compounds. Using this sequential “+/−”screening method, we were able to select compounds that are D₁ selective amongst the dopamine D₂, serotonin 5HT₂, and adrenergic β(1, 2) receptors. These 7 g-protein coupled receptors (GPCR) demonstrate significant sequence homology. We used a full-rank training matrix of 1,573 compounds×7 biological targets to build individual partitioning trees. Each “tree” was related to an individual target; all trees were built with the same compound set, unbiased towards any of the seven targets within the array.

From an initial library of 250,000 virtual compounds (obtained from commercial vendors and in the form of SD (digital-coded structure files) using the “positive leaves” of the Dopamine D₁-partitioning tree, we compiled a “long” list of compounds 40,000) that were statistically likely to be reactive with D₁ due to the presence of the “positive” descriptors. Since the targets share a significant sequence homology, reactivity of this list of compounds to the receptors within the array could not be excluded. However, this “long” list was further “trimmed” with the “negative leaves” of the six other “trees”. The “trimming” process used the “negative” nodes (leaves) to select compounds from the list of 40,000 compounds that already exhibited (in silico) likelihood of D₁ (T7) activity. Each “trimming” step afforded a smaller subset that was likely to be active against D_{I} and less likely to be active against another target in the set, since the list was “picked” using positive leaves of D₁ and negative leaves of the other trees. The final subset, much smaller than the original population, contained molecules, which had positive chemical descriptors for D₁ and negative descriptors for the other six targets. The list was then further “trimmed” using “Lipinsky's rule of five” for drug likeness and diversity assessments to afford a final 406-compound library, representing 1% of the original long list, or 0.16% of the original library of 250,000 virtual compounds. Finally, the 406 compounds selected via in silico studies were screened in the laboratory against the 7-target array at 10⁻⁵M. 34 compounds, representing 5 distinctly different chemical structural classes, exhibiting greater than 50% inhibitory activity for D₁ receptor were obtained. This constitutes a hit rate of 8.5% and demonstrates an 85-fold increase in hit rate (or productivity) as compared to the conventional screening of a random chemical library (hit rate of 0.1%). Moreover, 9 compounds showed nearly complete specificity for D₁ (activities are 5 fold more reactive with D₁ than with any others of the same array), and one compound exhibited a specific binding affinity in nM (Ki˜10⁻⁷M).

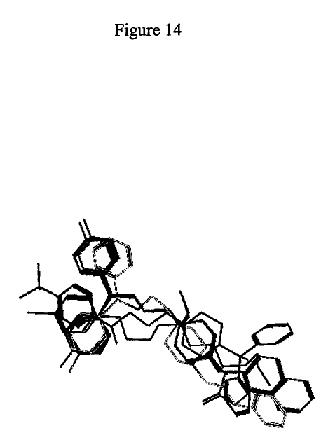

In short, this study demonstrates that “in silico probability differential screening” can be translated to actual in vitro selected reactivity or even target specificity in a given set of GPCR targets. This conclusion is reflected in a “landscape plot” represented in FIG. 13. The screening results of 406 compounds against 7 GPCR targets were plotted in a “pair-wise fashion”. The overall active compounds gravitate towards the axis representing dopamine D₁ binding activity; in addition 9 compounds demonstrate a near specific binding activity with dopamine D₁.

The development of the ion channel database described herein will enhance our knowledge of specific K⁺- and other ion channels as well. The proposed screening dataset and its gradual inclusion of pharmacological information of other ion channels, especially other K⁺-channels isoforms, provides a mechanism for systematic discovery of specific ion channel isoforms and agents which specifically modulate their activity.

Forecasting models (computational software and datasets) based on arrays of structure-activity relationships have been established between chemical descriptors and observed activity at an array of different binding sites (assays) on the HERG channel. The computational tools described herein, like any other screening tools, are not designed to replace the clinical monitoring of drug safety; instead they function as an assessment tool, like other screening methodology, for specific safety concerns.

As mentioned previously, E-4031, a potent HERG K+-channel inhibitor (observed functionally), did not demonstrate significant binding affinity in the astemizole directed binding assay. Thus, E-4031 “delivers” its effect at HERG protein at a site other than that bound by astemizole. Based on the chemical structures of E-4031, dofetilide and astemizole, and the pharmacological profiles of these agents, it appears that E-4031 binds to a region that “bridges” or overlaps a portion of the binding sites of dofetilide and astemizole. There is another reported peptide toxin binding site at the extracellular domain of the HERG K+-channel, which may affect K⁺-flux. Each of these sites will be further characterized using appropriate binding assays.

To identify all possible small molecule binding sites affecting channel activity other than those known sites relies on screening a substantial chemical library. Reportedly, there are 10⁷⁰ theoretically possible chemical entities (Valler and Green, 2000). Practically, there are about 1.5 to 3 million (10⁶) compounds available commercially and only about half of the compounds are considered to be of reasonable quality (purity and integrity) to be assessed in drug discovery methods.

We will select the 5 most reputable chemical venders, and ask each vendor to provide a selection of 2,000 to 2,500 diverse chemical compounds. These compounds will be compiled, with redundancy eliminated and triaged for drug-like properties using the Lipinski's rule of 5. Our initial goal is to attain a screening library of approximately 10,000 (10⁴; sampling of ˜1% of the population domain) compounds representing the commercially assessable chemical molecules. Screening this library against HERG-protein in a cell based functional assay will provide a seed dataset reflecting the domain of compounds where most of drug discovery is initiated; some of the “hits” may affect the ion channel activity from the known sites, others may act via different sites.

The entire compound collection (10,000+) will be tested for activity using DiBac₄ HTS assay (membrane potential dye) with the Flexstation. Due to the relatively low sensitivity of the assay, all compounds are tested for activities at 10⁻⁴M (100 μM) in duplicates. In an attempt to reduce false negatives, the substrate concentration will be about 10 to 100 fold higher than that of a conventional HTS.

Compounds indicating any activity in the cell base functional assay will be characterized initially in the three already developed radioligand binding assays, namely, astemizole, E-4031 and peptide-toxin bind assays. Those exhibiting binding affinity in any one of the three specific binding assays will be noted. Idiosyncrasies between the functional and binding assays, i.e. those that are showing functional effects yet without any “readings” from any site specific assays are likely molecules reacting with the sites other than those known. These molecules provide information regarding new and distinct binding sites.

Compounds exhibiting HERG functional activity without any indication of binding events against the established panel of binding assays will be tested for HERG protein “functional” activity (again) using detection methods of 1) atomic absorption and (if the compound fails to demonstrate activity) then with 2) path-clamping methods with the same recombinant cells in order to further confirm the initially observed functional activity and to eliminate potential false positives (perhaps due to the artifact of high substrate concentration) before committing to expansive isotopic labeling of chemical substrates. The most potent compound in functional assays will then be labeled with radioisotopes, e.g., ³H, to develop additional site-specific binding assays.

Any compound with demonstrated and confirmed activity will be used as a structural template to search for compounds sharing substructural components from the same commercial entities. These compounds will then be tested using the same panel of in vitro assays (bindings and functional), whereas those demonstrating confirmable activity will be used as structural guides and templates to identify additional similar compounds. Our experience in drug discovery has indicated that it is possible to carry out two to three such iterations with compounds (about 50 to 100 compounds) from commercial entities. With a sample size of 50 to 100 congeners with varying degree of activity, a sufficiently robust statistical model may be built based on the identified activity associated chemical descriptors.

As mentioned in Example 1, QSAR algorithms describe mathematic relationships between relevant chemical descriptors and the potencies of the observed biological activity, i.e. activity Y is a function of descriptorX, [Y=f(X)]. Chemical entities may be represented (described) by different chemical descriptors, either as sub-structural components or moieties, distance of chemical functional groups, or spatial, 2D or 3D topological, electrochemical, electro-physical, and or quantum mechanical properties of the small molecules. When different clusters of chemicals react with a protein at a specific site, some of these descriptors are found to be the contributing factors of the bimolecular interactions.

As set forth above, the QSAR algorithms of the invention used to predict potential HERG activity were generated using QSARIS, a canned software, tool package for building different QSARs. It provides users with different possibilities to “operate” with various sets of molecular descriptors, different regression algorithms and the coupled used of genetic algorithms (GA).

The program provides a default number of 250 chemical descriptors separated into 3 categories, 2D descriptors bearing structure information as 2 dimensional topological object (5 sub-categories, ˜200+descriptors); 3D descriptor, which is a set of physical properties based on quantum-mechanics and physicochemical calculation (2 sub-categories, 24 descriptors) and one general descriptor namely logP (a measure of a compounds distribution in water versus an organic solvent).

The program also provides different algorithms in data interrogations including ordinary multiple (OMR), stepwise (SWR), all possible subsets (PSR), and partial least squares (PLS) regressions and genetic algorithms (GA). Depending on the type (mostly the size) of the data, one may experiment with different combination of descriptors and algorithms to test and establish experimental models. These models are experimentally validated, i.e. testing compounds predicted active (inactive) in actual in vitro assays.

When dealing with a dataset of relatively small sample size, dependent-independent variables (numbers of hits) of the initial data, ordinary multiple regression (OMR) should be sufficient for data handling, yet it should not preclude the user from trying the other methods especially when the multicollinearity is unknown. We used OMR in Example 1 as it is the simplest method of the regression analysis. Ordinary Multiple Regression coupled GA computes the least squares fit in several independent variables (descriptors) to the dependent variable (% inhibitions). The form of the regression equation is a relationship of Y=bₒ+b₁·X₁+b₂·X₂+ . . . +bₚ·Xₚ; whereby Y represents %-inhibition (or potency) and X represent different chemical descriptors.

The selection of chemical descriptor is important for model building, i.e. supervised “learning” is required. The combination of different chemical descriptors best “representing” the set of compounds was experimentally determined using sets of 2-dimensional descriptors. The reason for using 2D descriptors is simply due to the numbers of descriptor available and their easy (comprehensible) link to medicinal chemistry.

As set forth in Example 1, 24 compounds exhibited different inhibitory potencies against the “activity” of HERG K⁺-channel. These potencies were then further characterized in parallel with three different experimental parameters: 1) binding, 2) whole cell functional with membrane potential dye and 3) with AA. We set binding affinity as the chemical's HERG K⁺-channel “activity” appreciating that the degree of binding affinity (potency) may or may not be equivalent to “functional” potency.

The size of the database produced in Example 1 approximates the size of a typical series of compounds one may find from an iterative screening process with compounds from a commercial source. That is, a typical screen of a diversified chemical library (with a redundancy of 2, only 2 similar compounds in a set), one may find active leads as singlets (hits without any others similar) or doublets (two structural similar hits). Using the structures of the “hits” as templates iteratively, one may collect a secondary (or the tertiary) focus library of 20 to 30 or more structural congeners.

We will employ different clustering methods, such as RP, which can be used upon completion of a substantial dataset. In this case, a substantial dataset generated from 1) binding assay and/or 2) functional assay will identify lead compounds representing different structural and classes of compounds. Our experience suggests that this will be a scattered and heterogeneous dataset and thus it will initially difficult to develop QSAR relationships. We will therefore enrich each compound for whatever chemical information it may “represent”. We will also enrich each compound or alternatively each cluster of compounds with additional analogues. We will also 1) enrich each cluster using the positive “leaves” from the partitioning tree to enrich each cluster with positive screening hits; and 2) using the RP clustered subset of the compounds in a regression model for QSAR construction. Thus, clustering-regression methods will also be used to augment the construction of our computation models

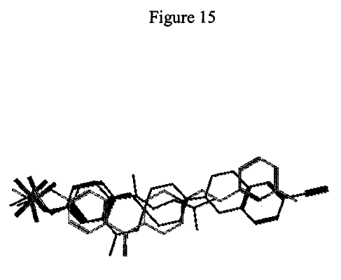

Compounds demonstrating consistent and relatively potent activity in all three assays were selected for further study. These included GBR12909, GBR12935, terfenadine, pimozide, sertindole, and clofilium. These compounds include common structural elements: 1) the nitrogen of the piperazinyl (GBR12909, GBR12935,) or piperidinyl (terfenadine, pimozide and sertindole) with one exception, clofilium, an tetra-alkyl ammonium group, and 2) the relative through-bond distance (˜5) of these nitrogen to the hydrophobic aromatic component of the molecule, which may be considered as putative pharmacophore with respect to HERG protein activity. As shown in FIG. 14, with GBR 12909 marked in green; GBR12935 in white; terfenadine in red; pimozide in grey, and clofilium in blue, the molecular alignment indicated that the distances between the ternary nitrogen (of the piperazines or piperidines) and the hydrophobic aromatic ring (or rings) 5 (or 4) bonds away from the nitrogen are the contributing factor in their consistent activities with the HERG K⁺-channel proteins, and the “4^{th}-atom” from the nitrogen (or the benzylic position) may be a SP³-carbon or a heteroatom of hydrogen bond donor or acceptor, such as —O— or —NH—. In fact, ten of the remaining eighteen compounds used in this study including amiodarone, impiramine, astemizole, cyproheptadine, diphenhydramine, clozapine, haloperidol, risperidone, verapamil, cisapride may also be “aligned” within the same SAR configurations. It appears that these 16 compounds represent a likely congruent small molecular orientation reflecting the binding site of HERG protein as represented by astemizole binding. This SAR observation is consistent with the 3-dimensional QSAR study published by the Lilly's group using Catalyst (Ekins, et al, 2002). That study reported that an important feature of small molecules demonstrating HERG protein binding activity is the distance of the hydrophobic sphere and the ionizable feature. This is consistent with the SAR described herein, that is, the ionizable group is equivalent to the ternary nitrogen, and the hydrophobic sphere is equivalent to the space occupied by the aromatic moieties.

With this SAR-model, however, it is still difficult to explain the lack of functional activity in the dye-based assay for nicardipine except that the 4^{th}-atom from the ternary nitrogen is SP² configuration (similar to E-4031) and the aromatic unit is not a conjugated benzyl.

Seven other compounds, cocaine, quinidine, ketoconazole, erythromycine, propranolol, E-4031 and sotalol do not appear to fit within the present SAR models. Regardless of what their “functional readings” may be (mostly active at least in one of the two functional assays), nearly all of them exhibited low binding affinity at the astemizole site. Certain of these compounds lack demonstrable affinity which may be attributable to a variety of factors, e.g., pH or temperature of the assays. Propranolol and quinidine activity appear to be affected by the pH conditions of the assay.

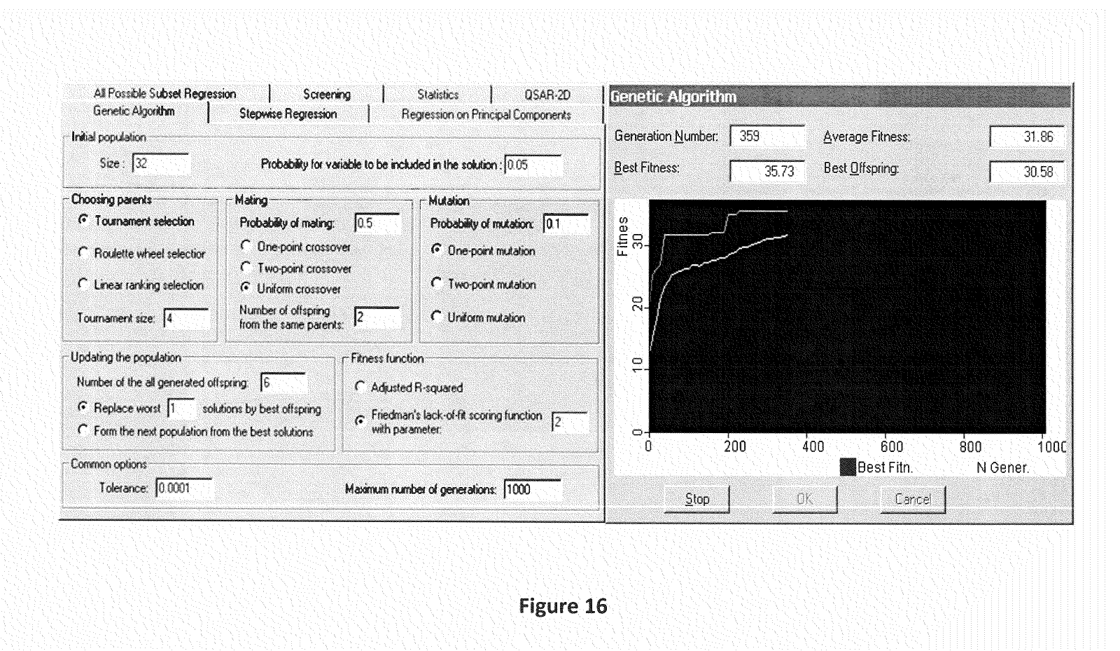

Interestingly, the results obtained with E-4031 and sotalol appear to indicate the existence of another HERG binding site. These two compounds belong to a family of “HERG K+-channel active” methanesulfonanilides, which include compounds like MK-499, (grey), included in FIG. 15. This observation is consistent with a recent study using alanine-scanning mutagenesis. Mitcheson et al (of Sanguinetti's group) report that “the binding site, corroborated with homology modeling, is comprised of amino acids located on the S6 transmembrane domain (G648, Y652, and F656) and pore helix (T623 and V625) of the HERG channel subunit that face the cavity of the channel. Terfenadine and cisapride interact with Y652 and F656, but high-affinity binding site for methanesulfonanilides may involve different amino acid residues” (Mitcheson et al, 2000). Since E-4031 consistently demonstrated potent functional activities in both functional formats, we putatively named this potential new site the E-4031-site.

Patch-clamp studies in HEK 293 cells show that both erythromycin and clarithromycin significantly inhibit HERG potassium current at clinically relevant concentrations. Erythromycin reduced the HERG encoded potassium current in a concentration dependent manner with an IC₅₀ of 38.9 μM. Clarithromycin produced a similar concentration-dependent block with an IC₅₀ of 45.7 μM (Stanat et al 2003). Similar observations were obtained using our functional assessments under appropriately modified experimental conditions. In another report, “mechanistic studies showed that inhibition of HERG current by clarithromycin did not require activation of the channel and was both voltage- and time-dependent. The blocking time course could be described by a first-order reaction between the drug and the channel. Both binding and unbinding processes appeared to speed up as the membrane was more depolarized, indicating that the drug-channel interaction may be affected by electrostatic responses” (Walter et al, 2002) which may indicate another site of molecule interaction other than those dominated by hydrophobic and or combination of hydrophobic and ionic interactions.

The binding sites of cocaine and ketoconazole as well as different clusters of related compounds at these sites will also be explored using chemical analogues and iterative binding and functional assay approaches.

In general, the structure (SAR) analysis of the screening dataset has produced interesting results. Information produced from this study, like the SAR studies of the compounds demonstrating consistent activities are directly relevant and provide the medicinal chemist with guidance for library design and candidate optimization. The analyses of the negative data and incongruity between data sets have produced insight on molecular interactions that can be extrapolated to other ion channel related biological and structural activities.

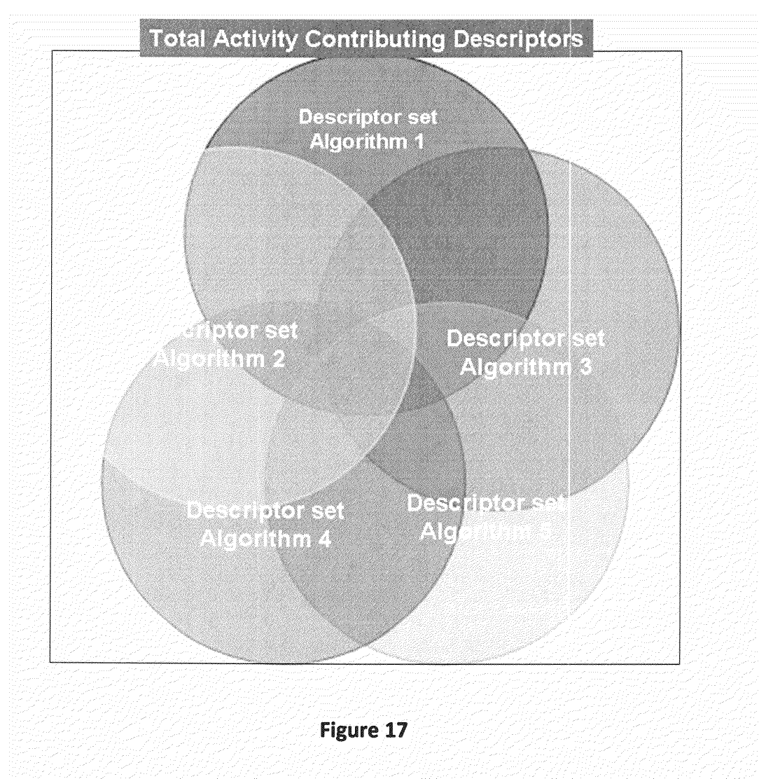

In recent years, genetic algorithms have been widely used for combinatorial optimization. Genetic algorithms (GA) use evolutionary operations to drive the process in computer-aided problem solving. The basic operations used here are random-mutation and genetic recombination (crossover) and their use leads to the optimization of solution of the predefined selection criteria. The difference of these methods from other search strategies is that they use a collection of intermediate solutions. These solutions are then used to construct new and hopefully improved solutions of the problem. Without going into great detail about the mathematic operations of the GA, FIG. 16 depicts a screen shot of GA in operation with QSARIS. In this software, GA is always used for the selection of optimal subset of descriptors followed with the selected statistical operations to establish the final correlation (QSAR algorithm). While GA selection was convenient, “human interference” is still necessary in order to uncover some less “obvious” factors which may nevertheless be important. Our initial operation in selecting different sets of subsets of chemicals descriptor in principle is to provide different starting points (initial population) of the evolutionary analysis.

Using the same data-handling techniques (consistent parameters GA coupled OMR) and same set of 2D-based structural descriptors, the binding dataset provided the most robust models demonstrated with high quality statistical parameters and cross-validation values. In this case, “INH=−22.18*SHBint2_Acnt+2.957E+004*xvch9+7.321*SaaCH_acnt−28.63*SaaN_acnt+24.52*Hmaxpos−50.3428 (eq.1)”.

The model emphasized the importance of two activity contributing factors: 1) hydrophobicity-aromaticity in terms of hydrocarbon valence, branching (2.957E+004*xvch9, topological chain/cluster counts, connectivity), and the total counts of aromatic hydrocarbons (7.321*SaaCH_acnt, E- tate); and 2) the maximum “ionizable” positive changes (24.52*Hmaxpos; E-state). All of these observations are consistent with the structural-activity relationship analysis; that is the importance of HERG activity is determined by the 1) the aromatic sphere (7.321*SaaCH_acnt), the ionizable positive changes of the nitrogen which may be protonated (24.52*Hmaxpos) and a defined distance between these two “factors” (partially described as in (2.957E+004*xvch9). Two other structural elements appear to be negatively affecting chemical interaction with HERG; one is inter-or intra-molecular hydrogen bonding which is consistent with our SAR studies with molecules able to form these bonds. Another factor is the total number of aromatic nitrogens.

The Rb-flux model may be improved by eliminating what we call as the statistical “over allotments”. In fact, this reflects an example of “human interference” in descriptor selection. As shown, the algorithm derived from the RB+-flux-AA detection method is initially described as “INH=−7.627*Gmin+766.6*xvch6−16.7*SdCH2+17.82*StsC 8.633*SsOH_acnt−14.254 (eq.2). There are two descriptors in this respective algorithm depicted to be positively (+17.82*StsC) and negatively (−16.7*SdCH₂) contributing to activity. When relating descriptors to the chemical-biological dataset, we identified that each descriptor is only represented by one molecule: SdCH₂, “═CH₂”, a moiety of the quinidine (#7); and StsC, “C≡N” moiety of the verapamil (#21).

When one (SDCH₂ for instance) of the two descriptors is “de-selected” (blocked, or removal from the descriptor table) from the panel of selected 130 descriptors, the data interrogation produced a significantly improved model: “INH=−41.09*SHBint3_Acnt−14.49*xp4+625.9*xvch6+2.83*k0+1.03*SHBint2+15.6723 (eq.3)”; with quality parameters like “Multiple R-Squared=0.9113; Standard error of estimation=11.11; F-statistic=34.95; P-value=2.299E-008; Multiple Q-Squared=0.8396; and Cross validation RSS=3799”. The analysis indicated that the training set is very well described by the regression equation, which is statistically very significant. Cross-validation shows that the constructed model can be used to predict the value of percent inhibition (INH) in this functional assay. Although the chemical descriptor included in this algorithm is not as directly apparent and comprehensible (to a medicinal chemist) as the previous one, it indicated the importance in hydrocarbon valance, branching and clusters (- 14.49*xp4+625.9*xvch6), and kappa zero index (information content and number of graph vertices etc). Note that the k0=I*(nvx), where nvx=number of graph y vertices, hydride groups and non-hydrogen atoms, a descriptor which will be seen in other experimental models from later experiments as well.

In one of the experiment, we choose to use only the combination of electro-topological state (E-state) indices and molecular properties including formula weight (fw), number of chemical elements in a molecule, number of graphic vertices (number of non-hydrogen atoms, number of hydride groups such as —CH₃, —OH etc; nvx), number of hydrogen bond acceptors and donors etc, which provided a panel of 44 different chemical descriptors. This set of chemical descriptors did not include the 2D connectivity components which the previous interrogation indicated to be important. Using the combined genetic algorithm and ordinary multiple regression, the computational program generated an algorithm: “INH=−11.26*numHBa−11.74*SssO_acnt+20.73*SsF_acnt−64.62*SddssS_acnt+12.26*SHBint8_Acnt+0.3362*fw−18.4559 (eq. 4)”. This algorithm weighted the contribution of the different hetero-atoms in the dataset, and is consistent with the chemistry observations. The binding affinity is likely associated with the size of the molecule (and may also be related to kappa indices, shape, in the previous model), to “fill” the respective binding cavities/crevices, hence the formula weight in positively contributing to the activity; the distended (8-bonds) intermolecular hydrogen bond may help to stabilize certain respective binding conformation, hence another positive positively contributing factor. For the descriptor SHBint8_Acnt, both astemizole and nicardipine “exhibited” possible internal hydrogen bonds with 8-bond distance. Sotalol and erythromycin also demonstrate the same possible internal hydrogen bonds, yet there are other factors that out-weigh the contribution of internal hydrogen bonds. For the highly oxygenated erythromycin, the sum of negative contribution of possible hydrogen bond acceptor and the total number of oxygen (−11.26*numHBa−11.74*SssO_acnt) greatly out weighted the positive contributions from the distended internal hydrogen bonds. For sotalol, the prominent negative contributing factor comes from the contributions of the sulfonamide (−64.62*SddssS_acnt). The contribution of “−F” (+20.73*SsF_acnt) accounts for the number of the potent inhibitors with the halogen substitutions.

The same data was further assessed by “blocking” the descriptor “fw” and extended internal hydrogen bond (≧8). The matrix was then reduced to a matrix of 37 E-state descriptors and 24 compounds with their respective inhibitory potencies; the resultant OMR algorithm indicated as “INH=2.678*nvx+6.632*SaaCH_acnt+32.06*SaaaC_acnt−53.97*SaaN_acnt−9.533*SssO_acnt−66.9227 (eq.5) Besides the numbers of the graphic vertices, “nvx” which are represented as numbers of non-hydrogen atoms and the number of hydride atoms (related to the size and weight of the molecules), the descriptors are depicted partially similar to the “models” previously discussed.

In addition to 2D chemical descriptors used to generate the above comparative models, we broadened the descriptor selection to include general molecular properties and property such as “cLogp” values; such an effort accounts for a model such as: “INH=11.31*LogP+204.4*xch6+1.806E+004*xvch9−43.29*SaaN_acnt−39.0381(eq.6)” with statistic quality parameter such “Multiple R-Squared=0.9069; Standard error of estimation=14.62; F-statistic=43.85; P-value=4.803E-009; Multiple Q-Squared=0.8149 and Cross validation RSS=7651”. With some of the similar “terms”, the training set is very well described by the regression equation, which is statistically significant. Cross-validation shows that the constructed model may be used to predict the percent inhibition. Comparing with the algorithm derived only with the 2D descriptor set (130 descriptors, eq.1), the value of logP sensibly replaced both the “accounts” of aromatic hydrocarbon and ionizable groups.

When we expanded the descriptor set to include 3D chemical descriptors, we used a different approach. The 2D to 3D structure conversion was carried out using Concord™ builder provided by the software. The descriptors are a set of physical properties calculated using different quantum-mechanical or physicochemical considerations. The default set of 3D descriptors is subdivided into two subgroups: 1) general—this is a set of 11 descriptors characterizing shape and dimensions of the molecule (surface, volume, and ovality), as well as atomic charges, dipole moments, and polarizabilities calculated using Gasteiger method; and 2) molecular moment—this is the set of 13 descriptors for Comparative Molecular Moment Analysis (CoMMA), which characterize absolute values and components of moments of inertia, dipole moment, and quadrupole moment of molecules. However, in contrast from the previous approach, when we include 3D descriptors in our data analysis, we started with same matrix of dataset, 24 compounds (=24 activity profiles)×(160, 2- and 3-D descriptor set), but the difference between each experiment is the selection of different “conditions” under which to afford the “genetic evolution”, i.e. different “parents”, mating “behaviors”, mutation ‘mechanism” and probabilities and maximum numbers of generation and offspring's. Amongst many iterations the OMR models appeared to be sufficiently robust, and with emphasis on a set of similar and dissimilar chemical descriptors, the following algorithms demonstrates the result of our experiments—1) INH=0.02316*Ix+6.044*SsssCH−5.182*SssO−27.9*SdsN_acnt−98.31*SddssS_acnt+12.65*ka3−5.9066 (eq.7) and 2) INH=−41.46*P−6.323*SssO−122.1*SddssS_acnt+12.72*ka3+2.537*Gmax+0.01082*Ix+71.43*Pz−1.65149 (eq. 8). Both algorithms provided sufficiently robust statistical parameters and cross-validations results so that the models are have utility in activity forecasting.

In conclusion, based on the statistical analyses, it is clear that the radioligand binding assay generated the most congruent and internally consistent set of data. The regression models depict arrays of chemical descriptors prominently affecting activity at the HERG K+ channel, which are also consistent with the structure-activity relationship.

With this dataset we have derived a panel (array) of algorithms from a large iteration of different computational experiments (≧80), each algorithm (model) depicting (weighting) a robust statistical relationship between different chemical descriptors and their respective combinations with the respectively observed activity (binding); the algorithm array represent a significant portion of the chemical descriptors affecting the chemical-HERG protein interactions, and effectively forecasts potential HERG activity at the astemizole binding site and other sites with reliability.

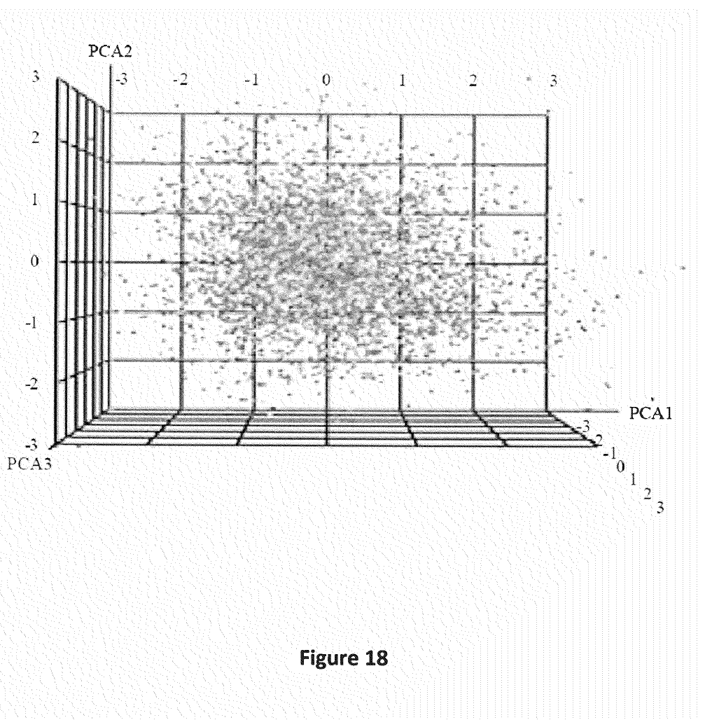

To test the validity (or the forecasting ability) of these QSAR models, we set up validation experiments. These experiments were designed to forecast or predict the activity of chemicals that are not in the training set, using the derived QSAR array, then testing the compounds (with predicted levels of activity) in the corresponding in vitro HERG binding assay. As shown previously, multiple QSAR algorithms are established, each depicting a different set of chemical descriptors. A schematic diagram of the algorithm combination is shown in FIG. 17.

These models were employed in scanning a chemical library of 2000 compounds, mostly medications, assay reference agents, or other previously known bioactive compounds. Forecasted inhibition of equal or greater than 50% is considered to be active. Compounds indicating 50% inhibition by all 5 models ( 5/5) concurrently are earmarked as “highly likely actives”; four of five models (⅘) are “likely actives”; three of five (⅗), maybe active; less than two of five (≦⅖), unlikely active.

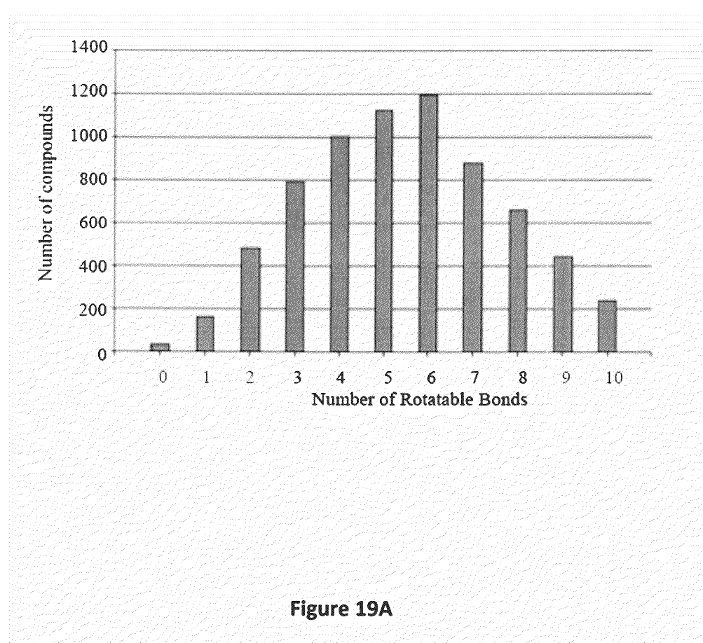

We will assess a diverse library of chemicals for interactions with HERG and other ion channels using a diverse set of compounds selected from our proprietary virtual database (compiled with different vendors SD Files of about 1 million entries). Descriptor clustering will be used with selection of drug-like criteria with computational tools such as DiverseSolutions (Tripos St. Louis Miss.). The graph in FIG. 18 represents a three dimensional principle component analysis of our recent selection of 7,030 compounds from 153,000 virtual structural files. The compounds were clustered based on 30 descriptors encoding topology, shape, size, polarizability and electrostatic parameters. To reduce the dimensionality, principal component analysis was used and clustering used to generate the 7,030 compound diversity set was based on 12 principal component analyses.

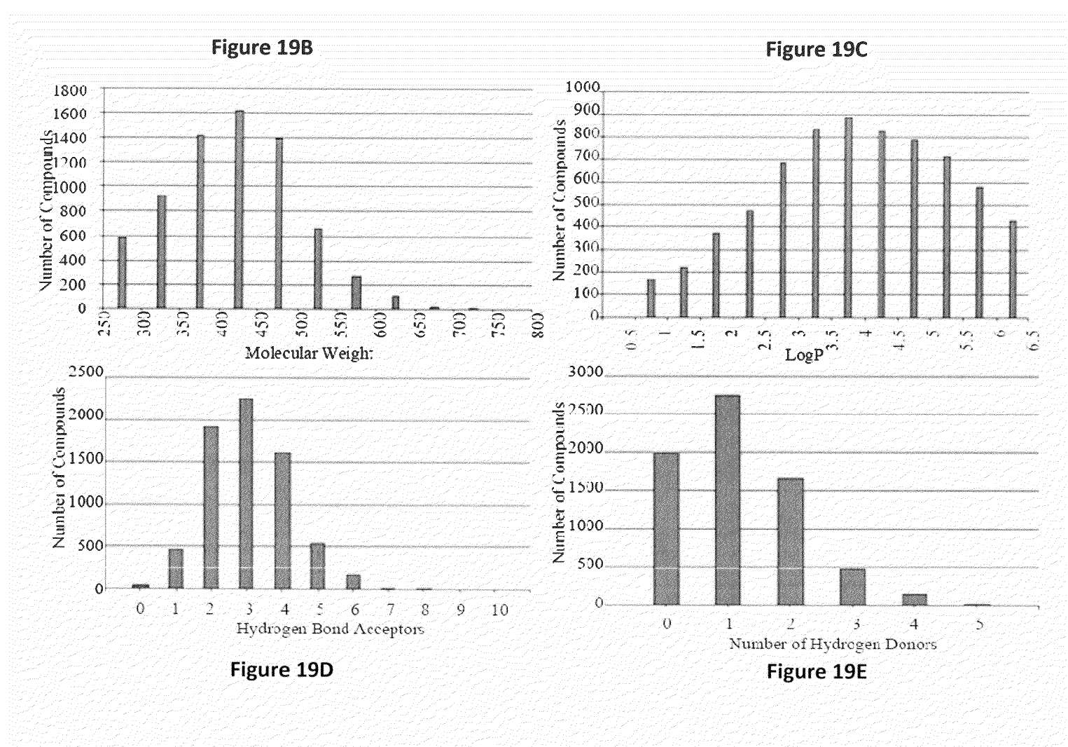

Medichem-rule and filters are used for such selection (of 7,030 entries) as in 1) molecule weights are between 250 to 800; 2) cLog between 0.5 to 6.5; 3) numbers of rotational bond 10; 4) numbers of heteroatoms 5 10 (data not shown); 5) hydrogen bond donors <5; and 6) H-bond acceptor ≦10. Additionally, undesired (unstable) chemical functionalities, such as —CHO, —COX, —OCOX, —COOOC—, —SH, NCO, NCS, SO₂X are visually eliminated. Consequently, the resultant 7,030 entities are with a distribution of molecular properties as indicated in FIGS. 19, panels a-e.

An identical process will be used with a large base of chemical structures (database) compiled from a selected group of vendors reasonably representing the accessible chemical space. We intend to collect (sample) approximately 10,000 compounds initially and 2) test these compounds in our screening programs for hits.

We will then perform hit-expansion analysis to expand the “population” of the hits identified from the biological assay thereby establishing robust and reliable forecasting models. The model is constructed statistically based on an appropriate number of samples indicating the statistical significance between the chemical descriptors and respective observed HERG K⁺-channel activity.

Agents which adversely impact potassium flux can lead to serious health consequences, including death. Table 4 provides a list of potassium channels which are suitable targets for the in silico screening methods of the invention.

TABLE 4

Kv1.1-1.8
Kv2.1-2.2
Kv3.1-3.4
Kv4.1-4.3
Kv5.1
Kv6.1-6.3
Kv7.1-7.5
Kv8.1
Kv9.1-9.3
Kv10.1-10.2
Kv11.1-11.3
Kv12.1-12.3
Kca1.1
Kca2.1-2.3
Kca3.1
Kca4.1-4.2
Kca5.1
Kir1.1
Kir2.1-2.4
Kir3.1-3.4
Kir4.1-4.2
Kir5.1
Kir6.1-6.2
Kir7.1
K2p1.1
K2p2.1
K2p3.1
K2p4.1
K2p5.1
K2p6.1
K2p7.1
K2p9.1
K2p10.1
K2p12.1
K2p13.1
K2p15.1
K2p16.1
K2p17.1
CNGA1-CNGA4
CNGB1
CNGB3
HCN1-HCN4
TRPC1-TRPC7
TRPV1-TRPV6
TRPM1-TRPM2
TRPM4
TRPM6-TRPM8

Code—code means all sequential numbers exist.

From “The IUPHAR Compendium of Voltage-gated Ion Channels” Edited by William A. Catterall, K. George Chandy and George A.Gutman Published 2002 by IUPHAR media

Certain known K⁺-channels ligands lack target specificity. Examples of such compounds are listed in Table 5. Most of these compounds are already part of the RSMDB collection and have been profiled for activities against a wide array of receptors, enzymes, transporters, and ion channels (Ca⁺⁺ and Na⁺ respectively. We will assess these compounds for interactions with the potassium ion channels listed above, including interactions with the HERG channel.

TABLE 5
Compounds of ion channel (K+)-related interests

1-ethyl-2-benzimidazoline
(1-EBIO)
5,8-diethoxypsoralen
Acetazolamide
Aflatrem
Almokalant
Ambasilide
Amitriptyline
Apamin
Aprikalim
Astemizole
Azimilide
Bepridil
BIIA 0388
Bimakalim
BMS-180448
BMS-189269
BMS-191095
BMS-204352
BRL32872
Bupivacaine
Capsaicin
Carbamazepine
Cetiedil
CGS7181
chlorpromazine
Chlorpropamide
chlorzoxazone
Chromanol 293B
Cisapride
Clamikalant (HMR 1883)
Clofilium
Clotrimazole
Clozapine
Cocaine
CP308408
CP-339,818
Cromakalim
Cyproheptadine
DCEBIO
Dequalinium
DHS-1
Diazoxide
Dilitazem
DMP 543
Dofetilide
D-sotalol
E-047/1
E-4031
Econazole
F3
Fampridine (4AP)
Flecainide
Glipizide
Glyburide
Halofantrine
haloperidol
halothane
HMR 1098
HMR 1556
HMR 1883
ibutilide
imipramine
isofluorane
ketoconazole
L735821
L-768673
linopirdine
Loratadine
Mefloquine
methylxanthine
minoxidil
MK-499
Nateglinide
Nicorandil
nifedipine
nimodipine
NDP
NIP121
NS 004
NS 1619
NS 8
NS1608
nitrendipine
Ondansetron
P 1075
Paxiline
Penitrem A
Pi1-NH
Pi1-OH
Pilocarpine
pimozide
Pinacidil
Pirenzepine
PNU-37883A
PNU83757
PO5
Quinidine
Repaglinide
retigabine
Riluzole
Rimakalim
RO 316930
Rupatadine
RWJ 29009
S 9947
SDZ 217 744
SDZ PCO 400
Sematilide
Sertindole
Sipatrigine
Symakalim
Tacrine
Tedisamil
terfenadine
tertiapin
thiopiridazine
Tolbutamide
TRAM-34
Trifluperasine
Tskappa
Tubocurarine
U 89232
UCL 1608
UCL 1684
UK 78,282
Verruculogen
WAY133537
WAY151616
WIN 17317-3
XE-991
YM-099
YM-934
ZD0947
ZD6169
ZM244985
Zoxazolamine

The majority of these compounds (hits) are obtainable from commercial venders of combinatorial chemistry. Additionally, there are many analogues available. According to our experience, with each hit, we can find approximately 30 to 50 analogues by substructural component analysis and or other category of chemical descriptors. Thus, to expand the “hit list”, we will acquire those that are similar and test for activity in the same array of assays as the second generation of focused (in contrast to diverse) chemical library to acquire sufficient data to be interrogated for statistical modeling.

We have described a process wherein we explored and interrogated the multi-dimensionalities of a robust dataset that reflect bi-molecular interactions at a specific site of a macromolecule. The process provided a robust set of quantitative SARs, each reflecting different statistical contributions of chemical descriptors and their combinations in respect to a relative binding affinity. As a matrix, these relationships provide a robust statistical forecasting model.

Using the new assays and approaches descrbied we should obtain a large and high density dataset. Initially, the entire dataset will be interrogated using clustering methods based on chemical descriptors such as 2-dimensional topological chemical descriptors described above along with recursive partitioning. It is noteworthy to point out that RP is not the only tool and algorithm available. At present, we have licensed the source-code (Java) from GoldenHelix (makers of ChemTree) for generating 2D topological chemical descriptors from mol and SD files. With this tool, we can generate an “interaction table” that links and associates both the molecules and their respective structural based descriptor to their respective biological activities. Other data handling methods, such as 1) “K-Means” by Forgy and Mc Queen algorithms (Hastie, 2001), a data handling technique popularized in gene array analysis (Corbeil et al, 2001; Fink, et al, 2003) which works well with large and “spotty” (missing data points) datasets, or 2) hybrid handling methods like HAC, which uses a combined approach to build the classification tree in two steps. We can (1) use a “fast” clustering method (K-Means) to produce many low-level clusters and (2) use these clusters for the dendogram construction (Wang, 1982); or 3) using a more “tedious” classification and regression algorithm (Radivojac et al 2004) with programs like DTREG (www.dtreg.comltechnical.htm) which interrogates well with small, dense and continuous datasets. The point of testing different data handling techniques provides further means to experimentally determine and to identify the “best possible” structural clusters (SAR clusters) which may be interrogated further for robust QSARs.

To de-convolute or to decipher different molecular binding sites we utilized combined functional and binding approaches, thereby separating high dimensional (heterogeneous and multiple site) “interactions” into smaller sets of site specific (lower dimensional) interactions using a biochemical assay approach, i.e. each lower dimensional data set reflecting a set of bimolecular interactions at a specific site of the macromolecule which could be more reliably handled and interrogated.

In short, we have developed reliable methods and systems for forecasting models of HERG protein interaction. Arrays of algorithms have been established that reflect mathematical relationships between the observed biological activity (with HERG protein) and essential chemical descriptors depicting chemical structure component(s) responsible to the observed activities. These algorithms are capable of ranking chemicals according to whether they possess potential reactivity with the HERG protein. Using these algorithms the medicinal chemist will be able to “scan” chemical libraries during compound acquisition (or library design process) or prior to conversion of a virtual chemical library to an actual one. For convenience, the algorithms should be implemented early in the library design process to avoid making compounds with apparent HERG-liability.

### REFERENCES DESCRIBING TEST COMPOUNDS UTILIZED IN EXAMPLES I and II DIPHENHYDRAMINE

1) Zareba W, et al., Electrocardiographic findings in patients with diphenhydramine overdose, Am J Cardiol, 1997 November, 80(9): 1168-73.
2) Wang W X, et al., “Conventional” antihistamines slow cardiac repolarization in isolated perfused (Langendorff) feline hearts, J Cardiovasc Pharmacol, 1998 July, 32(1): 123-8. Ergtoxin
3) Pardo-Lopez L, Garcia-Valdes J, Gurrola G B, Robertson G A, Possani L D, Mapping the receptor site for ergtoxin, a specific blocker of ERG channels, FEBS Lett, 2002 January, 10(1-2): 45-9. BeKm-1
4) Korolkova Y V, et al., New binding site on common molecular scaffold provides HERG channel specificity of scorpion toxin BeKm-1, J Biol Chem, 2002 November, 277(45): 43104-9.
5) Korolkova Y V, et al., An ERG channel inhibitor from the scorpion Buthus Eupeus, J Biol Chem, 2001 March, 276(1):9868-76. Verapamil
6) De Ponti F, Poluzzi E, Cavalli A, Recanatini M, Montanaro N, Safety of non-antiarrhythmic drugs that prolong the OT interval or induce torsade de pointes: an overview, Drug Saf, 2002, 25(4): 263-86.
7) Yang T, Snyders D, Roden D M, Drug block of I(kr): model systems and relevance to human arrythmias, J Cardiovasc Pharmacol, 2001 November, 38(5): 737-44.
8) Chouabe C, Drici M D, Romey G, Barhanin J, Effects of calcium channel blockers on cloned cardiac K+ channels Ikr and Iks, Therapie, 2000 January-February, 55(1): 195-202.
9) Waldegger S, et al., Effect of verapamil enantiomers and metabolites on cardiac K+ channels expressed I Xenopus oocytes, Cell Physiol Biochem, 1999, 9(2): 81-9.
10) Zhang S, Zhou Z, Gong Q, Makielski J C, January C T, Mechanism of block and identification of the verapamil binding domain to HERG potassium channels, Circ Res, 1999 May, 84(9): 989-98.
11) Chouabe C, Drici M D, Romey G, Barhanin J, Lazdunski M, HERG and KvLQT1/IsK, the cardiac K+ channels involved in long OT symdromes, are targets for calcium channel blockers, Mol Pharmacol, 1998 October, 54(4): 695-703. Sertindole
12) Kongsamut S, Kang J, Chen X L, Roehr J, Rampe D, A comparison of the receptor binding and HERG channel affinities for a series of antipsychotic drugs, Eur J Pharmacol, 2002 August, 450(1): 37-41.
13) Kang J, Chen X L, Rampe D, The antipsychotic drugs sertindole and pimozide block erg3,a human brain K+ channel, Biochem Biophys Res Commun, 2001 August, 286(3): 4999-504.
14) Rampe D, Murawsky M K, Grau J, Lewis E W, The antipsychotic agent sertindole is a high affinity antagonist of the human cardiac potassium channel HERG, J Pharmacol Exp Ther, 1998 August, 286(2): 788-93. Risperidone
12) Kongsamut S, Kang J, Chen X L, Roehr J, Rampe D, A comparison of the receptor binding and HERG channel affinities for a series of antipsychotic drugs, Eur J Pharmacol, 2002 August, 450(1): 37-41.
15) Ekins S, Crumb W J, Sarazan R D, Wikel J H, Wrighton S A, Three-dimensional quantitative structure-activity relationship for inhibition of human ether-a-go-go-related gene potassium channel, J Pharmacol, 2002 May., 301(2): 427-34. Pimozide
12) Kongsamut S, Kang J, Chen X L, Roehr J, Rampe D, A comparison of the receptor binding and HERG channel affinities for a series of antipsychotic drugs, Eur J Pharmacol, 2002 August, 450(1): 37-41.
16) Finlayson K, Turnball L, January C T, Sharkey J, Kelly J S, [3H]dofetilide binding to HERG transfected membranes: a potential high throughput preclinical screen, Eur J Pharmacol, 2001 October, 430(1): 147-8.
17) Osypenko V M, Degtiar Vie, Shuba IaM, Naid'onov V, Testosterone modulation of HERG potassium channel blockade induced by neuroleptics, Fiziol Zh, 2001, 47(3): 11-8.
18) Shuba M, Degtiar V E, Osipenko V N, Naidenov V G, Woosley R L, Testosterone-mediated modulation of HERG blockade by proarrhythmic agents, Biochem Pharmacol, 2001 July, 62(1): 41-9.
19) Osypenko V M, Degtiar Vie, Naid'onov V, Shuba IaM, Blockade of HERG K+ channels expressed in Xebopus oocytes by antipsychotic agents, Fiziol Zh,_{—}2001, 47(1): 17-25.
20) Kang J, Wang L, Cai F, Rampe D, High affinity blockade of the HERG cardiac K(+) channel by the neuroleptic pimozide, Eur J Pharmacol, 2000 March, 392(3): 137-40. Haloperidol
21) CNRS-UPR 411, Valbonne-France, Cardiac K+ channels and drug-acquired long QT syndrome, Therapie, 2000 January-February, 55(1): 185-93.
22) Suessbrich H, Schonherr R, Heinemann S H, Attali B, Lang F, Busch A E, The inhibitory effect of the antipsychotic drug haloperidol on HERG potassium channels expressed in Xenopus oocytes, Br J Pharmacol, 1997 March, 120(5): 968-74. Clozapine
23) Buckley N A, Sanders P, Cardiovascular adverse effects of antipsychotic drugs, Drug Saf, 2000 September, 23(3): 215-28. Erythromycin
24) Volberg W A, Koci B J, Su W, Lin J, Zhou J, Blockade of human cardiac potassium channel human ether-a-go-go-related gene (HERG) by macrolide antibiotics, J Pharmacol Exp Ther, 2002 July, 302(1): 320-7.
25) Bell I M, et al., 3-Aminopyrrolinone farnesyltransferase inhibitors: design of macrocyclic compounds with improved pharmacokinetics and excellent cell potency, J Med Chem, 2002 June, 45(12): 2388-409.
26) Butrous G, Siegel R L, Sildenafil (Viagra) prolongs cardiac repolarization by blocking the rapid component of the delayed rectifier potassium current, Circulation, 2001 June, 103(23): 119-20.
27) Henz B M, The pharmacologic profile of desloratadine, Allergy, 2001, 65: 7-13. Terfenadine
28) Scherer Cr, et al., The antihistamine fexofenadine does not affect I(Kr) currents in a case report of drug-induced cardiac arrhythmia, Br J Pharmacol, 2002 November, 137(6): 892-900.
29) Rajamani S, Anderson, C L, Anson B D, January C T, Pharmacological rescue of human K(+) channel long-QT2 mutations: human ether-a-go-go-related gene rescue without block, Circulation, 2002 June, 105(24): 2830-5.
30) Taglialatela M, et al., Inhibition of depolarization-induced [3H]noradrenaline release from SH-SY5Y human neuroblastoma cells by some second-generation H(1) receptor antagonists through blockade of store-operated Ca(²⁺) channels (SOCs), Biochem Pharmacol, 2001 November, 62(9): 1229-38.
31) Ducic I, Ko C M, Shuba Y, Morad M, Comparative effects of loratadine and terfenadine on cardiac K+ channels, J Cardiovasc Pharmacol, 1997 July, 30(1): 42-54. Cyproheptadine
32) Grzelewska-Rzymowska I, Pietrzkowicz M, Gorska M, The effect of second generation histamine antagonists on the heart, Pneumonol Alergol, 2001., 69(2-4): 217-26.
33) Kreutner W, Hey JA, Chiu P, Barnett A, Preclinical pharmacology of desloratadine, a selective and nonsedating histamine H1 receptor antagonist. 2^{nd} communication: lack of central nervous system and cardiovascular effects, Arzneimittelforschung, 2000 May, 50(5): 441-8.
34) Crumb W J Jr., Loratadine blockade of K(+) channels in human heart: comparison with terfenadine under physiological conditions, J Pharmacol Exp Ther, 2000 January, 292(1): 261-4.
35) Hey J A, Affrime M, Cobert B, Kreutner W, Cuss F M, Cardiovascular profile of loratadine, Clin Exp Allergy, 1999 July, 29(3): 197-9.
36) Taglialatela M, et al., Molecular basis for the lack of HERG K+]channel block-related cardiotoxicity by the H1 receptor blocker cetirizine compared with other second-generation antihistamines, Mol Pharmacol, 1998 July, 54(1): 113-21. Cisapride
37) Wang J, Della Penna K, Wang H, Karczewski J, Connolly T M, Koblan K S, Bennett P B, Salata J J, Functional and pharmacological properties of canine ERG potassium channels, Am J Physiol Heart Circ Physiol, 2003 January, 284(1): 256-67.
38) Paulussen A, Raes A, Matthijs G, Snyders D J, Cohen N, Aerssens J, A novel mutation (T65P) in the PAS domain of the human potassium channel HERG results in the long QT syndrome by trafficking deficiency, J Biol Chem, 2002 December, 277(50): 48610-6.
39) Chen J, Seebohm G, Sanguinetti MC, Position of aromatic residues in the S6 domain, not inactivation, dictates cisapride sensitivity of HERG and eag potassium channels, Proc Natl Acad Sci USA, 2002 September, 99(19): 12461-6.
40) Paakkari I, Cardiotoxicity of new antihistamines and cisapride, Toxicol Lett, 2002 February, 127(1-3): 279-84.
41) Benatar A, Cools F, Decraene T, Bougatef A, Vandenplas Y, The T wave as a marker of dispersion of ventricular repolarization in premature infants before and while on treatment with the I(Kr) channel blocker cisapride, Cardiol Young, 2002 January, 12(1): 32-6.
42) Potet F, Bouyssou T, Escande D, Baro I, Gastrointestinal prokinetic drugs have different affinity for the human cardiac human ether-a-gogo K(+) channel, J Pharmacol Exp Ther, 2001 December, 299(3):1007-12. Cocaine
43) Zhang S, et al., Cocaine blocks HERG, but not KvLQT1+mink, potassium channels, Mol Pharmacol, 2001 May, 59(5): 1069-76.
44) O'Leary M E, Inhibition of HERG potassium channels by cocaethylene: a metabolite of cocaine and ethanol, Cardiovasc Res., 2002 January, 52(1): 6-8.
45) Ferriera S, Crumb W J Jr, Carlton C G, Clarkson C W, Effects of cocaine and its major metabolic on the HERG-encoded potassium channel, J Pharmacol Exp Ther, 2001 October, 299(1): 220-6. Ketoconazole
46) Dumaine R, Roy M-L, Brown A M, Blockade of HERG and Kv1.5 by ketoconazole, J Pharmacol Exp Ther, 1998 286(2): 727-35. Imipramine
47) Teschemacher A G, Seward E P, Hancox J C, Witchel H J, Inhibition of the current of heterologously expressed HERG potassium channels by imipramine and amitriptyline, Br J Pharmacol, 1999 September, 128(2): 479-85. Amiodarone
48) Kiehn J, Thomas D, Karle C A, Schols W, Kubler W, Inhibitory effects of the class III antiarrhythmic drug amiodarone on cloned HERG potassium channels, Naunyn Schmiedebergs Arch Pharmacol, 1999 March, 359(3): 212-9.
49) Kamiya K, et al., Short- and long-term effects of amiodarone on the two components of cardiac delayed rectifier K(+) current, Circulation, 2001 March, 103(9): 1317-24. Quinidine
50) Paul A A, Witchel H J, Hancox J C, Inhibition of the current of heterologously expressed HERG potassium channels by flecainide and comparison with quinidine, propafenone and lignocaine, Br J Pharmacol, 2002 July, 136(5): 717-29.
51) Po S S, et al., Modulation of HERG potassium channels by extracellular magnesium and quinidine, J Cardiovasc Pharmacol, 1999 February, 33(2): 181-5. Sotalol
52) Numaguchi H, et al., Probing the interaction between inactivation gating and Dd-sotalol block of HERG, Circ Res, 2000 November, 87(11): 1012-8. E-4031
53) Spector P S, Curran M E, Keating M T, Sanguinetti M C, Class III antiarrhythmic drugs block HERG a human cardiac delayed rectifier K+channel. Open-channel block by methanesulfonanilides, Circ Res, 1996 March, 78(3): 499-503.
54) Wang S, Morales M J, Liu S, Strauss H C, Rasmusson R L, Modulation of HERG affinity for E-4031 by [K+]o and C-type inactivation, FEBS, 1997 November, 417(1): 43-7. Sertindole
55) Kang J, Chen X L, Wang L, Rampe D, Interactions of the antimalarial drug mefloquine with the human cardiac potassium channels KvLQT1/minK and HERG, J Pharmacol Exp Ther. 2001 October; 299(1):290-6. Astemizole
56) Taglialatela M, Pannaccione A, Castaldo P, Giorgio G, Annunziato L, Inhibition of HERG K(±) channels by the novel second-generation antihistamine mizolastine, Br J Pharmacol, 2000 November, 131(6): 1081-8.
57) Suessbrich H, Waldegger S, Lang F, Busch A E, Blockade of HERG channels expressed in Xenopus oocytes by the histamine receptor antagonists terfenadine and astemizole, FEBS Lett., 1996 Arril, 385(1-2): 77-80.
58) Zhou Z, Vorperian V R, Zhang S, January C T, Block of HERG potassium channels by the antihistamine astemizole and its metabolites desmethylastemizole and norastemizole, J Cardiovasc Electrophysiol, 1999 June, 10(6): 836-43.
59) Taglialatela M, et al., Cardiac ion channels and antihistamines: possible mechanisms of cardiotoxicity, Clin Exp Allergy, 1999 July, Suppl 3: 182-9. Clofilium
60) Suessbrich H, et al, Specific block of cloned Herg channels by clofilium and its tertiary analog LY97241, FEBS Letter, 1997, 414(2): 435-8. Other
61) Finlayson K, Pennington A J, Kelly J S, [³H]-dofetilide binding in SHSY5Y and HEK293 cells expressing a HERG-like K+ channel?, Eur J Pharmacol, 2001 February, 412(2): 203-12.
62) Yu S P, Kerchner G A, Endogenous voltage-gated potassium channels in human embryonic kidney (HEK293) cells, J Neurosci Res, 1998 52: 612-7.
63) Tang W, et al, Development and evaluation of high throughput functional assay methods for HERG potassium channel, J Biomol Screen, 2001 October, 6(5): 325-31.
64) Cui J, Melman Y, Palma E, Fishman G I, McDonald T V, Cyclic AMP regulates the HERG K(+) channel by dual pathways, Curr Biol, 2000 June, 10(11):671-4.
65) Lees-Miller J P, Duan Y, Teng G Q, Thorstad K, Duff H J, Novel gain-of-function mechanism in K(+) channel-related long-QT syndrome: altered atm and selectivity in the HERG1 N629D mutant, Circ Res, 2000 March, 86(5): 507-13.
66) Cavalli A, Poluzzi E, DePonti F, Recanatini M, Toward a pharmacophore for Drugs Inducin the Long QT Syndrome: Insights fraom a CoMFA Study of HERG K+ Channel Blockers, J Med Chem, 2002 July, 45:3844-53.
67) Catterall W. A., From ionic currents to molecular mechanisms: The structure and function of voltage-gated sodium channels, Neuron 2000, 26:13-25.
68) Belelli D., et al., General anaesthetic action at transmitter-gated inhibitory amino acid receptors, Trends Pharmaol. Sci. 1999, 20:496-502.
69) Sigel E., Buhr A., The benzodiazepine binding site of GABA, receptors, Trends Pharmocol. Sci. 1997, 18:425-429.
70) Maelicke A., Allosteric modulation ofnicotinic receptors as a treatment strategy for Alzheimer's disease, Dement GeriatrCogn Disord 2000 September, Suppl.1: 11-8.
71) Gray P W, Glaister D, Seeburg P H, Guidotti A, Costa E, Cloning and expression of a cDNA for human diazepam binding inhibitor, a natural ligand of an allosteric regulatory site of the gamma-aminobutyric acid type A receptor, Proc Natl Acad Sci USA 1986 October, 83(19):7547-51.
72) Roche O, et al, A Virtual Screening Method for Prediction of the hERG Potassium Channel Liability of Compound Libraries, ChemBioChem 2002, 3: 455-459.
73) Ekins S, et al, Three-Dimensional Quantitative Structure-Activity Relationship for Inhibition of Human Ether-a-Go-Go-Related Gene Potassium Channel, Jrnl Pharmacol Expl Ther. 2002, 301: 427-434. Propranolol
74)Kawakami K, Nagatomo T, Abe H, Kikuchi K, Takemasa H, Anson B D, Delisle B P, January C T, Nakashima Y. Comparison of HERG channel blocking effects of various beta-blockers—implication for clinical strategy. Br J Pharmacol. 2005 November 28; [Epub ahead of print]
75) Yao X, McIntyre M S, Lang D G, Song I H, Becherer J D, Hashim M A. Propranolol inhibits the human ether-a-go-go-related gene potassium channels. Eur J Pharmacol. 2005 September 20; 519(3):208-11.
76) Dupuis D S, Klaerke D A, Olesen S P Effect of beta-adrenoceptor blockers on human ether-a-go-go-related gene (HERG) potassium channels Basic Clin Pharmacol Toxicol. 2005 February; 96(2):123-30.
77) Chatrath R, Bell C M, Ackerman M J. Beta-blocker therapy failures in symptomatic probands with genotyped long-QT syndrome. Pediatr Cardiol. 2004 Sep-Oct; 25(5):459-65. Epub 2004 Jul. 30.
78) Imai T, Okamoto T, Yamamoto Y, Tanaka H, Koike K, Shigenobu K, Tanaka Y Effects of different types of K+ channel modulators on the spontaneous myogenic contraction of guinea-pig urinary bladder smooth muscle. Acta Physiol Scand. 2001 November; 173(3):323-33.

### REFERENCES FOR EXAMPLE 2

Angelo K, et al., A radiolabeled peptide ligand of the hERG channel, [¹²⁵I]-BeKm-1, Eur. J. Physiol 2003; 447: 55-63.
Barnard E. A., Langer S. Z., GABAA receptors; The IUPHAR Compendium of Receptor Characterization and Classification, 2^{nd} edition, IUPHAR Media, London UK, 2000, 104-110.
Berul C I, Morad M, Regulation of potassium channels by nonsedating antihistamines, Circulation 1995 Apr. 15; 91(8): 2220-5.
Cavalli A, Poluzzi E, DePonti F, Recanatini M, Toward a Pharmacophore for Drugs Inducing the Long QT Syndrome: Insights from a CoMFA Study of HERG K+ Channel Blockers, 2002; 45: 3844-3853.
Cheng Y, Prusoff W H, Relationship between the inhibition constant (K1) and the concentration of inhibitor which causes 50 per cent inhibition (I50) of an enzymatic reaction, Biochem Pharmacol 1973 Dec. 1; 22(23): 3099-108.
Cui J, Kagan A, Qin D, Mathew J, Melman Y F, McDonald T V, Analysis of the Cyclic Nucleotide binding domain of the HERG Potassium Channel and Interactions with KCNE2, J. Biol Chem 2001 May 18; 276(20): 17244-51.
Drici M D, Barhanin J, Cardiac K+ channels and drug-acquired long QT syndrome, Therapie 2000 January-February; 55(1): 185-93.
Ekins S, Crumb W, Sarazan R D, Wikel J H, Wrighton S A, Three-Dimensional Quantitative Structure-Activity Relationship for Inhibition of Human Ether-a-Go-Go-Related Gene Potassium Channel, J. Pharmacol Exp Ther, 2002; 310: 427-434.
Finlayson K, Pennington A J, Kelly J S, [³H]-dofetilide binding in SHSY5Y and HEK293 cells expressing a HERG-like K+ channel?, Eur. J Pharmacol 2001 Feb. 2; 412(3):203-212.
Heylen L, et al., Development of a HERG channel binding assay, Poster #2534, 2002 Society for Biomolecular Screening.
Isbrandt D, Friederich P, Solth A, Haverkamp W, Ebneth A, Borggrefe M, Funke M, Sauter K, Breithardt G, Pongs O, Schulze-Bahr E, Identification and functional characterization of a novel KCNE2 (MiRP1) mutation that alters HERG channel kinetics, J Mol Med 2002 August; 80(8): 524-32.
Jones-Hertzog D K, Mukhopadhyay P, Keefer C E, Young S S, Use of recursive partitioning in the sequential screening of G-protein-coupled receptors, J. Pharmacol Toxicol Methods 1999 December; 42(4): 207-15.
Kang J, Wang L, Cai F, Rampe D, High affinity blockade of the HERG cardiac K(+) channel by the neuroleptic pimozide, Eur J. Pharmacol 2000 Mar. 31; 392(3): 137-40.
Kiehn J, Thomas D, Karle C A, Schols W, Kubler W, Inhibitory effects of the class III antiarrhythmic drug amiodarone on cloned HERG potassium channels, Naunyn Schmiedebergs Arch Pharmacol 1999 March; 359(3): 212-9.
Kiss L, Bennett P, Uebele V, Koblan K, Kane S, Neagle B, Schroeder K, High Throughput Ion-Channel Pharmacology: Planar-Array-Based Voltage Clamp, Assay Drug Dev. Tech 2003; 1(1-2): 127-135.
Korolkova Y, Kozlov S, Lipkin A, Pluzhnikov K, Hadley J, Filippov A, Brown D, Angelo K, Strobaek D, Jespersen T, Olesen S, Jensen B, Grishin E, An ERG Channel Inhibitor from Scorpion Buthus eupeus, J Biol Chem 2001 March; 276 (13): 9868-986.
O'Leary M E, Inhibition of human ether-a-go-go potassium channels by cocaine, Mol Pharmacol 2001 February; 59(2): 269-277.
Po S S, Wang D W, Yang I C, Johnson J P Jr, Nie L, Bennett P B, Modulation of HERG potassium channels by extracellular magnesium and quinidine, J. Cardiovasc Pharmacol 1999 February ; 33(2): 181-5.
Rampe D, Murawsky M K, Grau J, Lewis E W, The Antipsychotic Agent Sertindole is a High Affinity Antagonist of the Human Cardiac Potassium Channel HERG, J. Pharmacol Exp Ther 1998 August; 286(2): 788-93.
Rampe D, Roy M L, Dennis A, Brown A M, A mechanism for the proarrhythmic effects of cisapride (Propulsid): high affinity blockade of the human cardiac potassium channel HERG, FEBS Lett 1997 November; 417(1): 28-32.
Smart T., et al. The nature reviews drug discovery ion channel questionnaire participants, Nature Rev Drug Disc, 2004 Mar., 3(3), 239-278.
Suessbrich H, Schonherr R, Heinemann S H, Lang F, Busch A E, Specific block of cloned Herg channels by clofilium and its tertiary analog LY97241, FEBS Lett 1997 Sep. 8; 414(2): 435-8.
Tinel N, Diochot S, Borsotto M, Lazdunski M, Barhanin J, KCNE2 confers background current characteristics to the cardiac KCNQ1 potassium channel, EMBO J. 2000 December; 19(23): 6326-30.
Tseng G, Ikr: The hERG Channel, J Mol Cell Cardiol 2001; 33 835-849.
Walker B D, Singleton C B, Bursill J A, Wyse K R, Valenzuela S M, Qiu M R, Breit S N, Campbell T J, Inhibition of the human ether-a-go-go-related gene (HERG) potassium channel by cisapride: affinity for open and inactivated states, Br. J. Pharmacol 1999 September; 128(2): 444-50.
Weerapura M, Nattel S, Chartier D, Caballero R, Hebert T E, A comparison of current carried by HERG, with and without coexpression of MiRP1, and the native rapid delayed rectifier current. Is MiRP1 the missing link?, JPhysiol 2002 April; 540(Pt. 1): 15-27.
Zhang S, Zhou Z, Gong Q, Makielski J, January C, Mechanism of Block and Identification of the Verapamil Binding Domain to HERG Potassium Channels, Circ. Res. 1999 Feb. 14; 84(9): 989-998.

While certain preferred embodiments of the present invention have been described and specifically exemplified above, it is not intended that the invention be limited to such embodiments. Various modifications may be made to the invention without departing from the scope and spirit thereof as set forth in the following claims.

1. A method for identifying test compounds which modulate potassium channel activity, comprising;
a) assembling a dataset of agents known to modulate potassium channel activity, wherein said dataset contains biophysical and structural features of said agents which include observed biological effects of said agents on potassium channel activity;
b) providing a series of algorithms which describe the interaction of said structural features with said potassium channel;
c) assessing the test compound for the presence or absence of the structural features of a) using the algorithms of b), thereby identifying test compounds sharing structural features with said agents which also modulate potassium channel activity.

2. A test compound identified by the method of claim 1.

3. The method of claim 1, wherein said potassium channel is selected from the group of channels provided in Table 4.

4. The method of claim 1, wherein said agents are selected from the group consisting of the agents listed in Table 5.

5. The method of claim 1, wherein said potassium channel is the HERG protein channel.

6. The method of claim 5, wherein said biophysical and structural features of said agents are selected from the group consisting of at least one of molecular weight, binding affinity for HERG, chemical descriptor of said agent, solubility, hydrophobicity, hydrophilicity, primary protein structure, secondary protein structure tertiary protein structure, and alterations in HERG expression levels

7. The method of claim 5, wherein said biological effects are selected from the group consisting of at least one of modulation of potassium flux, membrane depolarization, absence of HERG protein interaction, HERG channel blockage, agonist activity, antagonist activity,

8. The method of claim 5, comprising contacting HERG expressing cells with the compound identified in step c) and determining the effects of said test compound on HERG channel function as compared to
i) cells which do not express HERG;
ii) HERG expressing cells which had not been exposed to said test compound; and
iii) HERG expressing cells exposed to an agent known to modulate HERG.

9. The method of claim 8, wherein HERG function is assessed using Rb+ efflux assay, membrane potential dye assay, atomic adsorption functional assay and whole cell membrane binding with detectably labeled radioligands.

10. The method of claim 5, comprising detectably labeling the compound identified in step c) and conducting in vitro binding assays to determine the binding affinity of said compound for said HERG protein.

11. The method of claim 1, further comprising adding data obtained from functional assays conducted on the test compounds identified in step c) to the dataset of step a).

12. The method of claim 1, further comprising adding the data obtained from in vitro binding assays on the test compounds identified in step c) to the dataset of step a).

13. The method of claim 8, wherein said HERG expressing cells are Chinese hamster ovary cells.

14. The method of claim 9, wherein said radioligand is selected from the group of ligands provided in Table 1.

15. The method of claim 14, wherein said radioligand is [³H]-astemizole.

16. The method of claim 14, wherein said radioligand is [³H]-E4031.

17. The method of claim 1, wherein administration of said test agent to a patient is associated with adverse biological effects.

18. The method of claim 1, wherein administration of said test agent to a patient is associated with beneficial biological effects.

19. The method of claim 1, wherein said test compounds are obtained from a combinatorial chemical library.

20. The method of claim 19, further comprising optimizing the binding and modulation activities of test compounds identified in said combinatorial chemical library.

21. (canceled)

22. (canceled)

23. (canceled)

24. (canceled)

25. A functional cell based assay for identifying test compounds suspected of modulating HERG protein activity via interaction at the E4031 site, comprising:
a) contacting HERG expressing cells with said test compound and determining the effects of said test compound on HERG channel function as compared to
i) cells which do not express HERG;
ii) HERG expressing cells which had not been exposed to said test compound; and
iii) cells exposed to E4031.

26. The method of claim 25, wherein HERG function is assessed using Rb+ efflux assay, membrane potential dye assay, atomic adsorption functional assay and cell membrane binding with detectably labeled radioligands.

27. An in vitro assay for determining a test compound's binding affinity for the E-4031 site on HERG protein or a fragment thereof, comprising:
a) providing HERG protein or a fragment thereof;
b) detectably labeling a test compound which binds HERG at said E4031 site;
c) performing a competitive binding assay with said detectably labeled test compound in the presence and absence of test compound that has not been detectably labeled, thereby determining the binding affinity of said test compound for said 4031 site on said HERG protein.

28. (canceled)
